SEQUENCE LISTING (8 sequences)

SEQ ID NO: 1 - protein, 5 aa, synthetic construct
  REGION 1..5: note = source = /note="Description of Artificial Sequence: Synthetic peptide"
GGGGS

SEQ ID NO: 2 - protein, 10 aa, synthetic construct
  REGION 1..10: note = source = /note="Description of Artificial Sequence: Synthetic peptide"
GGGGSGGGGS

SEQ ID NO: 3 - protein, 330 aa, Homo sapiens [sequence omitted]

SEQ ID NO: 4 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 123 aa, synthetic construct
  REGION 1..123: note = source = /note="Description of Artificial Sequence: Synthetic polypeptide"
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 6 - protein, 107 aa, synthetic construct
  REGION 1..107: note = source = /note="Description of Artificial Sequence: Synthetic polypeptide"
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 7 - protein, 243 aa, synthetic construct
  REGION 1..243: note = source = /note="Description of Artificial Sequence: Synthetic polypeptide"
EVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSSGGGGSEVQLLESGGGLVQPGGSLRLSCAASGFTVSSYGMSWVRQAPGKGPEWVSGIRGSDGSTYYADSVKGRFTISRDNSKNTLYLQMNSLRAEDTAVYYCAKQGENDGPFDHRGQGTLVTVSS

SEQ ID NO: 8 - protein, 50 aa, synthetic construct
  SITE 1..50: note = /note="This sequence may encompass 1-10 'Gly Gly Gly Gly Ser' repeating units"
  REGION 1..50: note = source = /note="Description of Artificial Sequence: Synthetic polypeptide"
GGGGSGGGGSGGGGSGGGGSGGGGSGGGGSGGGGSGGGGSGGGGSGGGGS

(19) United States
(12) Patent Application Publication
(10) Pub. No.: US 2025/0197497 A1
(43) Pub. Date: Jun. 19, 2025

(54) METHODS FOR PURIFYING HETERODIMERIC, MULTISPECIFIC ANTIBODIES

(71) Applicant: TENEOBIO, INC., Thousand Oaks, CA (US)

(72) Inventors: Brett Jorgensen, Fremont, CA (US); Ute Schellenberger, Palo Alto, CA (US)

(21) Appl. No.: 19/030,905

(22) Filed: Jan. 17, 2025

Related U.S. Application Data
(63) Continuation of application No. 17/278,268, filed on Mar. 19, 2021.
(60) Provisional application No. 62/734,566, filed on Sep. 21, 2018.
(60) Provisional application No. 62/742,821, filed on Oct. 8, 2018.

(51) Int. Cl. C07K 16/28; B01D 15/38; C07K 1/22; C07K 16/30

(52) U.S. Cl. CPC C07K 16/2809; B01D 15/3809; C07K 1/22; C07K 16/30; B01D 15/3804; C07K 2317/31

(57) ABSTRACT

Methods for purifying heterodimeric, multispecific antibodies from solution are provided.

### CROSS-REFERENCE TO RELATED APPLICATIONS

This application claims priority benefit of the filing date of U.S. Provisional Patent Application No. 62/734,566, filed on Sep. 21, 2018, as well as U.S. Provisional Patent Application No. 62/742,821, filed on Oct. 8, 2018, the disclosures of which applications are herein incorporated by reference in their entireties.

### FIELD OF THE INVENTION

The present invention relates to methods for purifying heterodimeric, multispecific antibodies from solution.

### BACKGROUND OF THE INVENTION

Bispecific antibodies (BsAb) are an important new class of protein therapeutics. BsAb are designed to recognize and bind to two different antigens, often for the purpose of retargeting immune effector cells to kill cancer cells. Currently, two BsAb are approved as therapeutics by the European Medicines Agency (EMA) and one by the US Food and Drug Administration (FDA). Often during the purification of heterodimeric multispecific antibodies the use of conventional Protein A chromatography for capture is problematic due, in part, to the presence of Fc-containing product variants in the crude BsAb mixture. Further, multimeric proteins, such as antibodies, have a higher tendency to aggregate, contributing to significantly increased impurity levels. As such, there is a need to develop purification methods that effectively remove product-specific (aggregates or degradation products) and process related (media components, HCP, DNA, chromatographic media used in purification, endotoxins, viruses, etc.) impurities and yield sufficient amount of the correct and complete multispecific antibody. There are various methods known in the art for the purification of antibodies, however, there is still an unmet need for alternative chromatography processes which are capable of separating and purifying multispecific antibodies from aggregates and complexes which may form, for example, as a result of process-driven modifications or manufacturing conditions.

### SUMMARY OF THE INVENTION

Aspects of the invention involve methods for purifying a multispecific IgG antibody from a mixture by affinity chromatography, the methods comprising: immobilizing the multispecific IgG antibody from said mixture on a first affinity chromatography column having binding specificity to a heavy chain constant domain of said IgG antibody; and eluting the multispecific antibody from the first affinity chromatography column with an elution buffer comprising an anti-aggregation composition to purify the multispecific antibody from the mixture, wherein the anti-aggregation composition comprises one or more polyols.

In some embodiments, the one or more polyols are selected from the group consisting of: mannitol, glycerol, sucrose, trehalose, and combinations thereof. In some embodiments, the one or more polyols have a concentration that ranges from about 5% to about 25% w/v. In some embodiments, the one or more polyols comprise glycerol, having a concentration that ranges from about 5% to about 15% w/v. In some embodiments, the glycerol has a concentration of about 10% w/v. In some embodiments, the one or more polyols comprise sucrose, having a concentration that ranges from about 5% to about 15% w/v. In some embodiments, the sucrose has a concentration of about 10% w/v. In some embodiments, the elution buffer comprises about 10% glycerol and about 10% sucrose w/v.

In some embodiments, the affinity chromatography column comprises a protein A chromatography resin. In some embodiments, the elution buffer is selected from the group consisting of: citrate, acetate, acetic acid, 4-Morpholineethanesulfonate (MES), citrate-phosphate, succinate, and combinations thereof. In some embodiments, the elution buffer comprises citrate in a concentration that ranges from about 20 mM to about 30 mM. In some embodiments, the elution buffer comprises citrate in a concentration of about 25 mM. In some embodiments, the elution buffer has a pH that ranges from about 3.2 to about 4.2. In some embodiments, the elution buffer has a pH that ranges from about 3.4 to about 3.8. In some embodiments, the elution buffer has a pH of about 3.6. In some embodiments, the elution buffer comprises about 25 mM citrate, about 10% glycerol, and about 10% sucrose, and wherein the elution buffer has a pH of about 3.6.

In some embodiments, the affinity chromatography column comprises a domain-specific chromatography resin that binds to a CH1 domain of the IgG antibody. In some embodiments, the elution buffer comprises a buffer selected from the group consisting of: citrate, acetate, acetic acid, 4-Morpholineethanesulfonate (MES), citrate-phosphate, succinate, and combinations thereof. In some embodiments, the elution buffer comprises acetic acid in a concentration that ranges from about 45 mM to about 55 mM. In some embodiments, the elution buffer comprises acetic acid in a concentration of about 50 mM. In some embodiments, the elution buffer has a pH that ranges from about 3.4 to about 4.4. In some embodiments, the elution buffer has a pH that ranges from about 3.8 to about 4.2. In some embodiments, the elution buffer has a pH of about 4.0. In some embodiments, the elution buffer comprises about 50 mM acetic acid, about 10% glycerol, and about 10% sucrose, and wherein the elution buffer has a pH of about 4.0.

Aspects of the invention include methods of reducing aggregation of a multispecific IgG antibody in an elution pool from an affinity chromatography procedure, the methods comprising: immobilizing the multispecific IgG antibody on a protein A affinity chromatography column; and eluting the multispecific IgG antibody from the protein A affinity chromatography column with an elution buffer comprising 25 mM citrate, 10% glycerol, and 10% sucrose w/v, wherein the elution buffer has a pH of 3.6.

Aspects of the invention include methods of reducing aggregation of a multispecific IgG antibody in an elution pool from an affinity chromatography procedure, the methods comprising: immobilizing the multispecific IgG antibody on an affinity chromatography column comprising a domain-specific chromatography resin that has binding affinity to a CH1 domain of the multispecific IgG antibody; and eluting the multispecific IgG antibody from the affinity chromatography column with an elution buffer comprising 50 mM acetic acid, 10% glycerol and 10% sucrose, wherein the elution buffer has a pH of 4.0.

In some embodiments, the multispecific IgG antibody comprises a first and a second binding unit. In some embodiments, the first binding unit comprises a heavy chain variable region of a heavy chain-only antibody. In some embodiments, the second binding unit comprises a heavy chain variable region of an antibody and a light chain variable region of an antibody. In some embodiments, the first binding unit comprises a heavy chain variable region of a heavy chain-only antibody and the second binding unit comprises a heavy chain variable region of an antibody and a light chain variable region of an antibody.

In some embodiments, the first binding unit has binding affinity to a tumor-associated antigen. In some embodiments, the second binding unit has binding affinity to an effector cell. In some embodiments, the effector cell is a T cell. In some embodiments, the second binding unit has binding affinity to a CD3 protein on the T cell. In some embodiments, the multispecific IgG antibody is a bispecific IgG antibody.

These and further aspects will be further explained in the rest of the disclosure, including the Examples.

### BRIEF DESCRIPTION OF THE DRAWINGS

FIG. 1 depicts a BsAb molecule in accordance with some embodiments of the invention.

FIG. 2 depicts a non-limiting example of a BsAb. This depicted embodiment includes a CD3-binding arm and a TAA-binding arm comprising a first and a second VH domain. In the depicted embodiment, the first and second VH domains are identical, and both having binding affinity to the TAA.

FIG. 3 depicts active and inactive forms of the BsAb depicted in FIG. 2. The active form is a heterodimer (panel A), while the inactive forms include a TAA homodimer, a Half-Ab, a CD3 homodimer, excess light chain (LC), and aggregates.

FIG. 4 depicts a graph of absorbance units (AU) as a function of time for an SEC analysis. The graph shows that a BsAb heterodimer is similar in size to the CD3 homodimer that only contains the CD3-binding arm (depicted in FIG. 3, panel B).

FIG. 5 depicts an IEF gel analysis showing that a BsAb heterodimer, a CD3 homodimer, and a TAA homodimer have different isoelectric points (pIs).

FIG. 6 depicts an elution profile from a Protein A chromatography column at pH 3.6. The result shows that the eluted peak is 96% of the total integrated area. Loading, equilibration and elution conditions are described.

FIG. 7 depicts a graph of absorbance units (AU) as a function of time for an SEC analysis, demonstrating that the BsAb aggregates after Protein A elution at pH 3.6. Buffer and flow rate conditions are described.

FIG. 8 depicts an SDS-PAGE analysis confirming that high molecular weight fractions correspond to the BsAb product.

FIG. 9 shows a series of graphs demonstrating that additives can reduce aggregation of Protein A eluted BsAb. The additives investigated included mannitol, glycerol, sucrose and trehalose, in various combinations.

FIG. 10, panel A, depicts an active BsAb molecule comprising a CH1 domain, and panel B depicts an inactive TAA homodimer.

FIG. 11 depicts an SDS-PAGE analysis comparing a Protein A pool and a CaptureSelect CH1 (CH1-XL) pool. The analysis demonstrates that the TAA homodimer is present in the CH1-XL flow through.

FIG. 12 is a comparison of a Protein A capture and elution profile (panel A) and a CH1-XL capture and elution profile (panel B) for a BsAb. The Protein A elution was conducted at a pH of 3.3, whereas the CH1-XL elution was conducted at a pH of 4.6.

FIG. 13 depicts a CH1-XL capture and elution profile of a BsAb, where the elution was conducted at pH 4. The results demonstrate that the BsAb eluted efficiently, representing 93% of the integrated peak area.

FIG. 14 depicts a graph of absorbance units (AU) as a function of time for an SEC analysis, demonstrating that BsAb eluted from CH1-XL contains minimal aggregates. The CH1-XL pool contained low HMW content (2.2%) with efficient product binding out of the harvested cell culture fluid (HCCF).

FIG. 15 is a table showing residence time and dynamic binding capacity of the CH1-XL chromatography resin. The results demonstrate the dynamic binding capacity (DBC) plateaus at 4 minutes (9.3 mg/mL).

FIG. 16 is a flow diagram illustrating the various upstream and downstream unit operations involved with the manufacturing process of a BsAb.

FIG. 17 depicts an SDS-PAGE analysis of the BsAb purification process.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); Harlow and Lane, Antibodies: A Laboratory Manual, Cold Spring Harbor Laboratory; (1988); and Uwe Gottschalk, “Process Scale Purification of Antibodies” (2017).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

The term “binding unit” as used herein refers to a polypeptide comprising at least one variable domain sequence (VH) that binds to a binding target, with or without an associated antibody light chain variable domain (VL) sequence. In some embodiments, a binding unit comprises a single VH domain of a heavy chain-only antibody. In other embodiments, a binding unit comprises a VH domain and a VL domain.

A “purified” antibody (e.g., a bispecific antibody) means that the antibody has been increased in purity, such that it exists in a form that is more pure than it exists in its natural environment and/or when initially synthesized and/or amplified under laboratory conditions. Purity is a relative term and does not necessarily mean absolute purity. The terms “purifying” or “separating,” as used interchangeably herein, refer to increasing the degree of purity of a desired molecule (such as a multispecific antibody, e.g., a bispecific antibody) from a composition or sample comprising the desired molecule and one or more impurities. Typically, the degree of purity of the desired molecule is increased by removing (completely or partially) at least one impurity from the composition.

Antibodies that can be purified according to methods of the invention include multi-specific antibodies. Multi-specific antibodies have more than one binding specificity. The term “multi-specific” or “multispecific” specifically includes “bispecific” and “trispecific,” as well as higher-order independent specific binding affinities, such as higher-order polyepitopic specificity, as well as tetravalent antibodies and antibody fragments. “Multi-specific” antibodies specifically include antibodies comprising a combination of different binding entities as well as antibodies comprising more than one of the same binding entity. The terms “multi-specific antibody,” “multi-specific heavy chain-only antibody,” “multi-specific heavy chain antibody,” and “multi-specific UniAb™” are used herein in the broadest sense and cover all antibodies with more than one binding specificity. In a non-limiting example, the multi-specific antibodies purified according to the present invention specifically include antibodies immunospecifically binding to a CD3 protein, such as a human CD3 and a BCMA protein, such as human BCMA.

As used herein, the term “aggregates” refers to protein aggregates, e.g., homodimers. It encompasses multimers (such as dimers, tetramers or higher order aggregates) of the multispecific antibodies, and/or subunits thereof, to be purified, and may result in, e.g., high molecular weight aggregates.

“Anti-aggregation composition” as used herein refers to a composition that reduces unwanted association of two or more proteins, e.g., multispecific antibodies, or subunits thereof. In some embodiments, an anti-aggregation composition comprises one or more polyols.

A “polyol” is a substance with multiple hydroxyl groups, and includes sugars (reducing and non-reducing sugars), sugar alcohols and sugar acids. Non-limiting examples of polyols include mannitol, glycerol, sucrose, trehalose, and sorbitol.

“Loading density” refers to the amount, e.g., in grams, of a composition put in contact with a volume of chromatography material, e.g., in liters. In some examples, loading density is expressed in g/L.

A “sample” refers to a small portion of a larger quantity of material. Generally, testing according to the methods described herein is performed on a sample. The sample is typically obtained from a “mixture,” which comprises a recombinant polypeptide preparation obtained, for example, from cultured recombinant polypeptide-expressing cell lines, also referred to herein as “product cell lines,” or from cultured host cells. A sample may be obtained from a mixture comprising, for example but not limited to, harvested cell culture fluid, from an in-process pool at a certain step in a purification process, or from the final purified product. The sample may also include diluents, buffers, detergents, and contaminating species, debris and the like that are found mixed with the desired molecule (such as a multispecific antibody, e.g., a bispecific antibody).

As used herein, “host cells” do not contain genes for the expression of recombinant polypeptides of interest or products, but rather, serve as a receptive host for such genes to be introduced, for example, by transfection.

The term “product” as described herein is the substance to be purified by the methods of the invention; for example, a polypeptide (e.g., a multispecific antibody).

The terms “heavy chain-only antibody,” and “heavy-chain antibody” are used interchangeably herein and refer, in the broadest sense, to antibodies lacking the light chain of a conventional antibody. Heavy chain-only antibodies are described for example in WO 2018/119215, the disclosure of which is incorporated by reference herein in its entirety.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies in accordance with the present invention can be made by the hybridoma method first described by Kohler et al. (1975) Nature 256:495, an can also be made via recombinant protein production methods (see, e.g., U.S. Pat. No. 4,816,567), for example.

An “intact antibody chain” as used herein is one comprising a full length variable region and a full length constant region (Fe). An intact “conventional” antibody comprises an intact light chain and an intact heavy chain, as well as a light chain constant domain (CL) and heavy chain constant domains, CH1, hinge, CH2 and CH3 for secreted IgG. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fe (constant domain) of their heavy chains, antibodies and various antigen-binding proteins from the IgG class can be provided as different subclasses. The IgG class of antibodies can be further divided into four “subclasses” (isotypes), e.g., IgG1, IgG2, IgG3, and IgG4. The Fc constant domains that correspond to the IgG class of antibodies may be referenced as γ (gamma). The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called κ and λ, based on the amino acid sequences of their constant domains. Methods in accordance with embodiments of the invention can be used with IgG antibodies of any subclass, i.e., IgG1, IgG2, IgG3 or IgG4, including variant sequences thereof (described further herein).

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; FcγRIIB1; FcγRIIB2; FcγRIIIA; FcγRIIIB receptors, and the low affinity FcRn receptor; and can be assessed using various assays known in the art. A “dead” or “silenced” Fe is one that has been mutated to retain activity with respect to, for example, prolonging serum half-life, but which does not activate a high affinity Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fe sequences may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604). The human IgG1 amino acid sequence (UniProtKB No. P01857) is provided herein as SEQ ID NO: 43. The human IgG4 amino acid sequence (UniProtKB No. P01861) is provided herein as SEQ ID NO: 44. Silenced IgG1 is described, for example, in Boesch, A. W., et al., “Highly parallel characterization of IgG Fe binding interactions.” MAbs, 2014. 6(4): p. 915-27, the disclosure of which is incorporated herein by reference in its entirety.

Other Fe variants are possible, including, without limitation, one in which a region capable of forming a disulfide bond is deleted, or in which certain amino acid residues are eliminated at the N-terminal end of a native Fe, or a methionine residue is added thereto. Thus, in some embodiments, one or more Fc portions of a binding compound can comprise one or more mutations in the hinge region to eliminate disulfide bonding. In yet another embodiment, the hinge region of an Fc can be removed entirely. In still another embodiment, a binding compound can comprise an Fe variant.

Further, an Fe variant can be constructed to remove or substantially reduce effector functions by substituting (mutating), deleting or adding amino acid residues to effect complement binding or Fc receptor binding. For example, and not limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

The Fe may be in the form of having native sugar chains, increased sugar chains compared to a native form or decreased sugar chains compared to the native form, or may be in an aglycosylated or deglycosylated form. The increase, decrease, removal or other modification of the sugar chains may be achieved by methods common in the art, such as a chemical method, an enzymatic method or by expressing it in a genetically engineered production cell line. Such cell lines can include microorganisms, e.g., Pichia Pastoris, and mammalian cell lines, e.g. CHO cells, that naturally express glycosylating enzymes. Further, microorganisms or cells can be engineered to express glycosylating enzymes, or can be rendered unable to express glycosylation enzymes (See e.g., Hamilton, et al., Science, 313:1441 (2006); Kanda, et al, J. Biotechnology, 130:300 (2007); Kitagawa, et al., J. Biol. Chem., 269 (27): 17872 (1994); Ujita-Lee et al., J. Biol. Chem., 264 (23): 13848 (1989); Imai-Nishiya, et al, BMC Biotechnology 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies expressed by these engineered cells are thus sialylated by the exogenous gene product. A further method for obtaining Fe molecules having a modified amount of sugar residues compared to a plurality of native molecules includes separating said plurality of molecules into glycosylated and non-glycosylated fractions, for example, using lectin affinity chromatography (See, e.g., WO 07/117505). The presence of particular glycosylation moieties has been shown to alter the function of immunoglobulins. For example, the removal of sugar chains from an Fe molecule results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (See, e.g., Kaneko, et al, Science 313:760 (2006)), whereas removal of fucose from the IgG leads to enhanced ADCC activity (See, e.g., Shoj-Hosaka, et al, J. Biochem., 140:777 (2006)).

In alternative embodiments, binding coumpounds purified according to the invention may have an Fe sequence with enhanced effector functions, e.g., by increasing their binding capacities to FcγRIIIA and increasing ADCC activity. For example, fucose attached to the N-linked glycan at Asn-297 of Fc sterically hinders the interaction of Fc with FcγRIIIA, and removal of fucose by glyco-engineering can increase the binding to FcγRIIIA, which translates into >50-fold higher ADCC activity compared with wild type IgG1 controls. Protein engineering, through amino acid mutations in the Fc portion of IgG1, has generated multiple variants that increase the affinity of Fe binding to FcγRIIIA. Notably, the triple alanine mutant S298A/E333A/K334A displays 2-fold increase binding to FcγRIIIA and ADCC function. S239D/1332E (2X) and S239D/1332E/A330L (3X) variants have a significant increase in binding affinity to FcγRIIIA and augmentation of ADCC capacity in vitro and in vivo. Other Fe variants identified by yeast display also showed the improved binding to FcγRIIIA and enhanced tumor cell killing in mouse xenograft models. See, e.g., Liu et al. (2014) JBC 289(6):3571-90, herein specifically incorporated by reference.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region according to this invention can comprise an antibody with or without K447.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9. Non-limiting examples of such linkers include GGGGS (SEQ ID NO: 1) (n=1) and GGGGSGGGGS (SEQ ID NO: 2) (n=2). Other suitable linkers can also be used, and are described, for example, in Chen et al., Adv Drug Deliv Rev. 2013 Oct. 15; 65(10): 1357-69, the disclosure of which is incorporated herein by reference in its entirety.

The term “bispecific three-chain antibody like molecule” or “TCA” is used herein to refer to antibody-like molecules comprising, consisting essentially of, or consisting of three polypeptide subunits, two of which comprise, consist essentially of, or consist of one heavy and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and an antigen binding domain that binds an epitope of a second antigen or a different epitope of the first antigen, where such binding domain is derived from or has sequence identity with the variable region of an antibody heavy or light chain. Parts of such variable region may be encoded by VH and/or VL gene segments, D and JH gene segments, or JL gene segments. The variable region may be encoded by rearranged VHDJH, VLDJH, VHJL, or VLJL gene segments.

A TCA binding compound makes use of a “heavy chain only antibody” or “heavy chain antibody” or “heavy chain polypeptide” which, as used herein, mean a single chain antibody comprising heavy chain constant regions CH2 and/or CH3 and/or CH4 but no CH1 domain. In one embodiment, the heavy chain antibody is composed of an antigen-binding domain, at least part of a hinge region and CH2 and CH3 domains. In another embodiment, the heavy chain antibody is composed of an antigen-binding domain, at least part of a hinge region and a CH2 domain. In a further embodiment, the heavy chain antibody is composed of an antigen-binding domain, at least part of a hinge region and a CH3 domain. Heavy chain antibodies in which the CH2 and/or CH3 domain is truncated are also included herein. In a further embodiment the heavy chain is composed of an antigen binding domain, and at least one CH (CH1, CH2, CH3, or CH4) domain but no hinge region. The heavy chain only antibody can be in the form of a dimer, in which two heavy chains are disulfide bonded other otherwise covalently or non-covalently attached with each other, and can optionally include an asymmetric interface between one or more of the CH domains to facilitate proper pairing between polypeptide chains. The heavy-chain antibody to be purified in accordance with embodiments of the invention belongs to the IgG class. In a particular embodiment, the heavy chain antibody is of the IgG1, IgG2, IgG3, or IgG4 subclasss, in particular the IgG1 subtype or the IgG4 subtype, including variants thereof (further described hererin).

An “epitope” is the site on the surface of an antigen molecule to which an antigen-binding region of a binding compound binds. Generally, an antigen has several or many different epitopes, and reacts with many different binding compounds (e.g., many different antibodies). The term specifically includes linear epitopes and conformational epitopes.

The term “valent” as used herein refers to a specified number of binding sites in an antibody molecule or binding compound.

A “multi-valent” binding compound has two or more binding sites. Thus, the terms “bivalent”, “trivalent”, and “tetravalent” refer to the presence of two binding sites, three binding sites, and four binding sites, respectively. Thus, a bispecific antibody purified by a method according to the invention is at least bivalent and may be trivalent, tetravalent, or otherwise multi-valent. A large variety of methods and protein configurations are known and used for the preparation of bispecific monoclonal antibodies (BsMAB), tri-specific antibodies, and the like.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell, such as a natural killer cell, is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T cell receptors or FcRs and perform effector functions. Preferably, the cells express at least FcγRIII and perform ADCC effector function. Examples of human leukocytes which mediate ADCC include natural killer (NK) cells, monocytes, cytotoxic T cells and neutrophils; with NK cells being preferred. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B cells and T cells including cytolytic T cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al., J. Immunol. Methods 202:163 (1996), may be performed.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, and/or individuals with autoimmune diseases, and the like. Subjects may be human, but also include other mammals, particularly those mammals useful as laboratory models for human disease, e.g., mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomeriation), clipping/hydrolysis/fragmentation (e.g., hinge region fragmentation), succinimide formation, unpaired cysteine(s), N-terminal extension, C-terminal processing, glycosylation differences, etc.

### II. Detailed Description

### Methods of Purification of Multispecific Antibodies

Often during the purification of heterodimeric multispecific antibodies, including, e.g., bispecific antibodies (BsAbs), the use of Protein A chromatography for capture is problematic due to the presence of unwanted Fc-containing product variants (e.g., unwanted homodimer species) in the crude BsAb mixture. In addition, multimeric proteins, such as antibodies, have a higher tendency to aggregate, contributing to significantly increased impurity levels. In designing a manufacturing process for BsAbs, alternative affinity methods for capture are therefore required. Properties and performance characteristics of Protein A chromatography unit operations, as well as alternative capture methods, are discussed herein.

Methods in accordance with embodiments of the invention involve purifying a multispecific antibody from a mixture using an affinity chromatography procedure, comprising contacting a first affinity chromatography column with the mixture, immobilizing the multispecific antibody on the first affinity chromatography column, contacting the first affinity chromatography column with an elution buffer, wherein the elution buffer comprises an anti-aggregation composition, and eluting the multispecific antibody from the first affinity chromatography column to purify the multispecific antibody from the mixture.

In other aspects, the invention provides methods of reducing aggregation of a multispecific antibody in an elution pool from an affinity chromatography procedure comprising contacting a protein A affinity chromatography column with a mixture comprising the multispecific antibody, immobilizing the multispecific antibody on the protein A affinity chromatography column, contacting the protein A affinity chromatography column with an elution buffer, wherein the elution buffer comprises 25 mM citrate, 10% glycerol, and 10% sucrose w/v, and wherein the elution buffer has a pH of 3.6, and eluting the multispecific antibody from the protein A affinity chromatography column to purify the multispecific antibody from the mixture.

In yet other aspects, the invention provides methods of reducing aggregation of a multispecific antibody in an elution pool from an affinity chromatography procedure comprising contacting an affinity chromatography column comprising a domain-specific chromatography resin which binds to a CH1 domain of an IgG antibody with a mixture comprising the multispecific antibody, immobilizing the multispecific antibody on the affinity chromatography column comprising the domain-specific chromatography resin, contacting the affinity chromatography column comprising the domain-specific chromatography resin with an elution buffer, wherein the elution buffer comprises 50 mM acetic acid, 10% glycerol and 10% sucrose, and wherein the elution buffer has a pH of 4.0, eluting the multispecific antibody from the affinity chromatography column comprising the domain-specific chromatography resin to purify the multispecific antibody from the mixture.

The methods of the invention can be used to purify multispecific antibodies comprising a plurality of binding units. In certain embodiments, the multispecific antibody, e.g., bispecific antibody, comprises comprises a first and a second binding unit. In some embodiments, the first binding unit comprises a heavy chain variable region of a heavy chain-only antibody. In some embodiments, the second binding unit comprises a heavy chain variable region of an antibody and a light chain variable region of an antibody. In certain embodiments, the multispecific antibody comprises a first binding unit comprising a heavy chain variable region of a heavy chain-only antibody and a second binding unit comprising a heavy chain variable region of an antibody and a light chain variable region of an antibody. In certain embodiments, the multispecific antibody is a heavy chain-only antibody. Heavy chain-only antibodies are described for example in WO 2018/119215, the disclosure of which is incorporated by reference herein in its entirety.

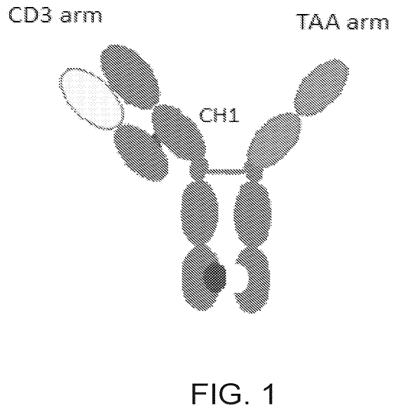

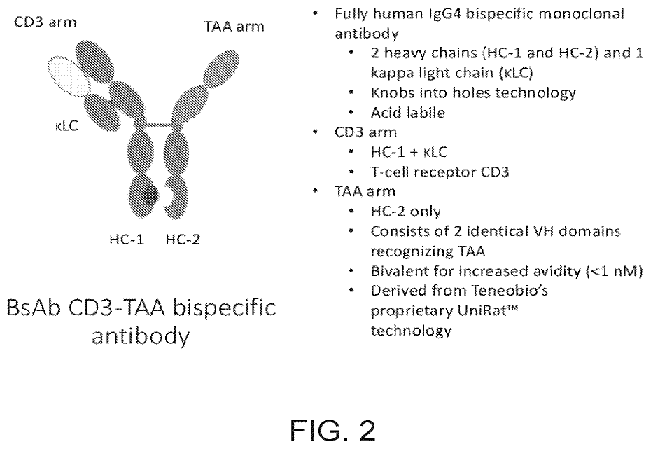

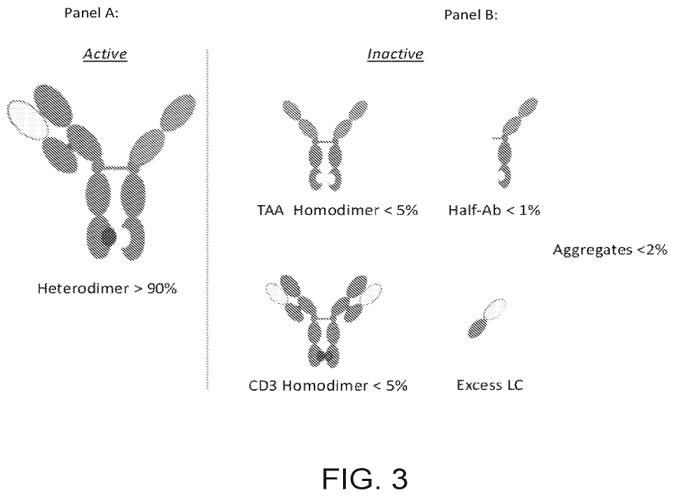

In certain embodiments, the multispecific antibody is a bispecific antibody. In some embodiments, a BsAb is an IgG type antibody, from any subclass (e.g., IgG1, IgG2, IgG3, IgG4), including engineered subclasses with altered Fc regions that provide for reduced or enhanced effector function activity. BsAbs in accordance with embodiments of the invention can be derived from any species. In one aspect, a BsAb is of largely human origin. In some embodiments, a BsAb is an IgG4 subtype, and is directed against a tumor associated antigen (TAA) in combination with CD3 (CD3-TAA). Non-limiting examples of BsAb in accordance with embodiments of the invention are depicted in FIG. 1 and FIG. 2. Inactive and active species are depicted in FIG. 3.

In some embodiments, the first binding unit of any of the multispecific antibodies described herein, e.g., bispecific antibodies, binds a tumor-associated antigen (TAA). Tumor-associated antigens (TAAs) are relatively restricted to tumor cells, whereas tumor-specific antigens (TSAs) are unique to tumor cells. TSAs and TAAs typically are portions of intracellular molecules expressed on the cell surface as part of the major histocompatibility complex. Non-limiting examples of tumor-associated antigens include CD38, CD19, CD22, and BCMA. In certain embodiments, the the second binding unit of any of the multispecific antibodies described herein binds an effector cell. In some embodiments, the effector cell is a T cell. In certain embodiments, the second binding unit binds CD3.

The term “CD3” refers to the human CD3 protein multi-subunit complex. The CD3 protein multi-subunit complex is composed to 6 distinctive polypeptide chains. These include a CD3γ chain (SwissProt P09693), a CD3δ chain (SwissProt P04234), two CD3ε chains (SwissProt P07766), and one CD3ζ chain homodimer (SwissProt 20963), and which is associated with the T cell receptor α and β chain. The term “CD3” includes any CD3 variant, isoform and species homolog which is naturally expressed by cells (including T cells) or can be expressed on cells transfected with genes or cDNA encoding those polypeptides, unless noted.

The term “BCMA” as used herein refers B-cell maturation antigen, also known as BCMA, CD269, and TNFRSF17, which is a member of the tumor necrosis receptor superfamily that is preferentially expressed in differentiated plasma cells. The term “human BCMA” as used herein includes any variants, isoforms and species homologs of human BCMA (UniProt Q02223), regardless of its source or mode of preparation. Thus, “human BCMA” includes human BCMA naturally expressed by cells, and BCMA expressed on cells transfected with the human BCMA gene.

In some embodiments, a BsAb is structurally a trimer, in which one arm (e.g., a CD3-binding arm) contains both fully human heavy and light chains, while the other arm (e.g., a TAA arm), derived from UniRat™ technology, consists of a human heavy chain (with one or more VH domains fused directly into a CH domain (comprising, e.g., hinge-CH2-CH3, and lacking a CH1 domain). Due to the unique structure of this BsAb, only the heterodimeric product contains a CH1 domain of human heavy chain (part of the CD3-binding arm).

The term “CD38” as used herein refers to a single-pass type II transmembrane protein with ectoenzymatic activities, also known as ADP-ribosyl cyclase/cyclic ADP-ribose hydrolase 1. The term “CD38” includes a CD38 protein of any human or non-human animal species, and specifically includes human CD38 as well as CD38 of non-human mammals. The term “human CD38” as used herein includes any variants, isoforms and species homologs of human CD38 (UniProt P28907), regardless of its source or mode of preparation. Thus, “human CD38” includes human CD38 naturally expressed by cells, and CD38 expressed on cells transfected with the human CD38 gene. The terms “anti-CD38 heavy chain-only antibody,” “CD38 heavy chain-only antibody,” “anti-CD38 heavy-chain antibody” and “CD38 heavy-chain antibody” are used herein interchangeably to refer to a heavy chain-only antibody as hereinabove defined, immunospecifically binding to CD38, including human CD38, as hereinabove defined. The definition includes, without limitation, human heavy chain antibodies produced by transgenic animals, such as transgenic rats or transgenic mice expressing human immunoglobulin, including UniRats™ producing human anti-CD38 UniAb™ antibodies, as hereinabove defined.

The terms “CD19” and “cluster of differentiation 19” as used herein refer to a molecule expressed during all phases of B cell development until terminal differentiation into plasma cells. The term “CD19” includes a CD19 protein of any human and non-human animal species, and specifically includes human CD19 as well as CD19 of non-human mammals. The term “human CD19” as used herein includes any variants, isoforms and species homologs of human CD19 (UniProt P15391), regardless of its source or mode of preparation. Thus, “human CD19” includes human CD19 naturally expressed by cells and CD19 expressed on cells transfected with the human CD19 gene. The terms “anti-CD19 heavy chain-only antibody,” “CD19 heavy chain-only antibody,” “anti-CD19 heavy chain antibody” and “CD19 heavy chain antibody” are used herein interchangeably to refer to a heavy chain-only antibody as hereinabove defined, immunospecifically binding to CD19, including human CD19, as hereinabove defined. The definition includes, without limitation, human heavy chain antibodies produced by transgenic animals, such as transgenic rats or transgenic mice expressing human immunoglobulin, including UniRats™ producing human anti-CD19 UniAb™ antibodies, as hereinabove defined.

The terms “CD22” and “cluster of differentiation-22” as used herein refer to a molecule belonging to the SIGLEC family of lectins, found on the surface of mature B cells, and to a lesser extent on some immature B cells. The term “CD22” includes a CD22 protein of any human and non-human animal species, and specifically includes human CD22 as well as CD22 of non-human mammals. The term “human CD22” as used herein includes any variants, isoforms and species homologs of human CD22 (UniProt P20273), regardless of its source or mode of preparation. Thus, “human CD22” includes human CD22 naturally expressed by cells and CD22 expressed on cells transfected with the human CD22 gene. The terms “anti-CD22 heavy chain-only antibody,” “CD22 heavy chain-only antibody,” “anti-CD22 heavy chain antibody” and “CD22 heavy chain antibody” are used herein interchangeably to refer to a heavy chain-only antibody as hereinabove defined, immunospecifically binding to CD22, including human CD22, as hereinabove defined. The definition includes, without limitation, human heavy chain antibodies produced by transgenic animals, such as transgenic rats or transgenic mice expressing human immunoglobulin, including UniRats™ producing human anti-CD22 UniAb™ antibodies, as hereinabove defined.

Non-limiting examples of other bispecific antibodies that can be purified using methods in accordance with embodiments of the invention include: blinatumomab (CD19×CD3, Amgen); catumaxomab (EpCAM×CD3, Trion Pharma); emicizumab (Factor IXa×Factor IX, Roche, Chugai); ABT-981 (IL-1alpha×IL-1beta, AbbVie); AFM13 (CD30×CD16a, Affimed); istiratumab (IGF-1R×HER3, Merrimack Pharmaceuticals); SAR156597 (IL-4×IL-13s, Sanofi); MP0250 (VEGF×HGF, Molecular Partners); MCLA-128 (HER3×HER3, Merus); MCLA-117 (CLEC12A×CD3, Merus); ALX-0761 (IL-17A×IL-17F, Ablynx); AMG 570 (BAFF×ICOSL, Amgen); AMG 211 (CEA×CD3, Amgen/MedImmune); AMG 330 (CD33×CD3, Amgen); AMG 420 (BCMA×CD3, Amgen); ABT-165 (DLL×VEGF, AbbVie); AFM11 (CD19×CD3, Affimed); MEDI4276 (HER2×HER2, AstraZeneca/Medlmmune); JNJ-61178104 (Johnson & Johnson/Genmab (targets not disclosed)); JNJ-61186372 (EGFR×cMET, Johnson & Johnson/Genmab); MDG006 (CD123×CD3, Macrogenics); MGD007 (gpA33×CD3, Macrogenics); duvortuxizumab (MDGO11) (CD19×CD3, Macrogenics/Johnson & Johnson); MDG009 (B7-H3×CD3, Macrogenics); MDGO10 (CD32B×CD79B, Macrogenics); REGN1979 (CD20×CD3, Regeneron); RG7386 (FAP×DR5, Roche); RG7828 (CD20×CD3, Roche/Genentech); RG7802 (CEA×CD3, Roche); RG7992 (FGFR1×KLB, Roche/Genentech); XmAbl4045 (CD123×CD3, Xencor/Novartis); and JNJ-63709178 (CD123×CD3, Johnson & Johnson/Genmab).

### Mixtures

Aspects of the present invention include methods for purifying multispecific antibodies from a mixture comprising the multispecific antibody and one or more contaminants using an affinity chromatography procedure. The mixture is generally one resulting from the recombinant production of the multispecific antibody, for example, from cultured recombinant polypeptide-expressing cell lines or from cultured host cells. A sample or mixture may be obtained from, for example but not limited to, harvested cell culture fluid (HCCF), from an in-process pool at a certain step in a purification process, or from the final purified product. The sample may also include diluents, buffers, detergents, and contaminating species, debris and the like that are found mixed with the desired molecule (such as a multispecific antibody, e.g., a bispecific antibody).

For recombinant production of the polypeptide, the nucleic acid encoding it is isolated and inserted into a replicable vector for further cloning (amplification of the DNA) or for expression. DNA encoding the polypeptide is readily isolated and sequenced using conventional procedures (e.g., where the polypeptide is an antibody by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence (e.g. as described in U.S. Pat. No. 5,534,615, specifically incorporated herein by reference).

Suitable host cells for cloning or expressing the DNA in the vectors herein are the prokaryote, yeast, or higher eukaryotic cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Examples of useful mammalian host cell lines include, but are not limited to, monkey kidney CV1 cells transformed by SV40 (COS-7, ATCC CRL 1651); human embryonic kidney cells (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and human hepatoma cells (Hep G2).

Host cells are transformed with the above-described expression or cloning vectors for polypeptide production and cultured in conventional nutrient media modified as appropriate for inducing promoters, selecting transformants, or amplifying the genes encoding the desired sequences.

The host cells used to produce the polypeptide of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, a polypeptide can be produced intracellularly, in the periplasmic space, or directly secreted into the medium. If a polypeptide is produced intracellularly, as a first step, the particulate debris, either host cells or lysed cells (e.g., resulting from homogenization), is removed, for example, by centrifugation or ultrafiltration. Where a polypeptide is secreted into the medium, supernatants from such expression systems are generally first concentrated using a commercially available protein concentration filter, for example, an Amicon or Millipore Pellicon ultrafiltration unit.

In certain embodiments, the multispecific antibody mixture is subjected to detergent treatment prior to purification comprising affinity chromatography. The multispecific antibody mixture is then subjected to one or more purification steps as described herein.

### Affinity Chromatography

In designing the purification process described herein, it was discovered that some BsAbs tend to aggregate when exposed to low pH. Thus, use of Protein A chromatography, with elution at low pH, was particularly problematic in such instances, stimulating investigation of affinity resins that could serve as suitable alternatives to Protein A. In spite of the reduced levels of aggregation observed from including additives in the Protein A elution buffer, as described herein, co-purification of unwanted homodimer species (e.g., TAA homodimer species) was still observed. In addition, the acid lability of the BsAb precludes use of low pH viral inactivation unit operations. Thus, methods in accordance with embodiments of the invention include chromatography unit operations that employ various types of affinity resins, including, but not limited to, Protein A affinity resin. In certain embodiments, the Protein A elution buffer is supplemented with an anti-aggregation composition, as described herein, to reduce unwanted homodimer aggregates in the eluate.

Other aspects of the invention include chromatography unit operations that employ affinity chromatography comprising a domain-specific chromatography resin that binds to a CH1 domain of an IgG antibody, and that selectively binds the heterodimeric multispecific antibody product over the heavy chain homodimer as a process impurity. In certain embodiments, the domain-specific chromatography resin is a CaptureSelect™ affinity resin. In some embodiments, the the domain-specific chromatography resin is CaptureSelect™ CH1-XL affinity resin.

Affinity chromatography resins or materials allow for the affinity-based retention of antibodies on a chromatographic support. Examples of affinity chromatography include, but are not limited to, e.g., protein A chromatography, protein G chromatography, protein A/G chromatography, or protein L chromatography. Examples of affinity chromatography material include, but are not limited to, ProSep®-vA, ProSep® Ultra Plus, Protein A Sepharose® Fast Flow, Toyopearl® AF-rProtein A, MabSelect™, MabSelect SuRe™, MabSelect SuRe™ LX, KappaSelect, CaptureSelect™, CaptureSelect™ FcXL, and CaptureSelect™ CH1-XL. In certain embodiments, the affinity chromatography material is provided in the form of a column. In certain embodiments, the affinity chromatography is performed in “bind and elute mode” (alternatively referred to as a “bind and elute process”). “Bind and elute mode” refers to a product separation technique in which a product (such as the multispecific antibody) in the sample binds the affinity chromatography material and is subsequently eluted from the affinity chromatography material. In some embodiments, the elution is a step elution, in which the composition of the mobile phase is changed stepwise, at one or several occasions, during the elution process. In certain embodiments, the elution is gradient elution, in which the composition of the mobile phase is changed continuously during the elution process.

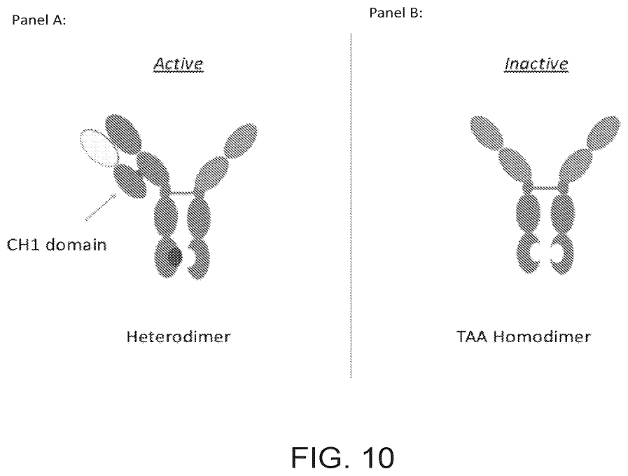

The general properties of the CH1-XL chromatography resin are that it comprises an Ig heavy chain CH1-specific nanobody ligand; it recognizes all four subclasses of IgG (i.e., IgG1, IgG2, IgG3 and IgG4); it is ligand immobilized on agarose having a size of 65 μm; it has a binding capacity of less than 20 mg/mL of IgG; it can be used under flow rate conditions of 5-200 cm/hr; it is stable to base (25-50 mM NaOH) for sanitization; and is commercially available. For purposes of purifying BsAb, the CH1-XL resin binds to bispecific heterodimer comprising a CH1 domain, but does not bind to the heavy chain homodimer species (e.g., the TAA homodimer). As shown in FIG. 10, only the active species includes a CH1 domain. In addition, the CH1-XL resin can be used under less stringent acidic elution conditions (pH 4). These gentler elution conditions contribute to reduced antibody aggregation in the elution pool.

“Load” refers to the composition being loaded onto a chromatography material. Loading buffer is the buffer used to load the composition (e.g., a composition comprising a multispecific antibody and an impurity or a composition comprising an antibody arm and an impurity) onto a chromatography material (such as any one of the chromatography materials described herein). The chromatography material may be equilibrated with an equilibration buffer prior to loading the composition which is to be purified. The wash buffer is used after loading the composition onto a chromatography material. An elution buffer is used to elute the polypeptide of interest from the solid phase.

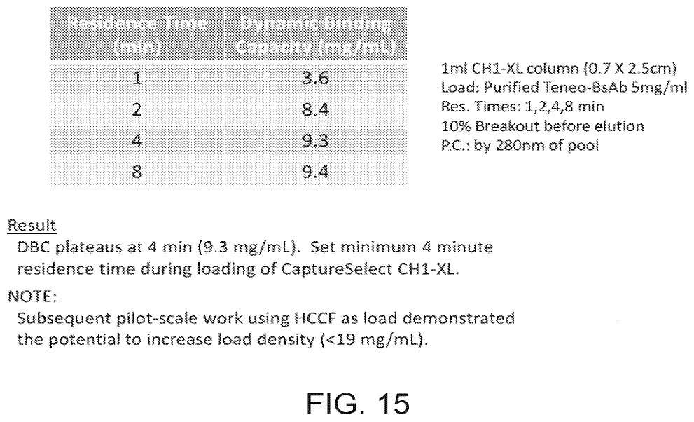

In some embodiments, the multispecific antibody composition is loaded onto an affinity chromatography material (e.g., a Protein A chromatography material, a CaptureSelect™ CH1-XL chromatography material) at a loading density of the multispecific antibody of about 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, or 19 mg/mL. Dynamic binding capacity (DBC) of the CH1-XL resin was investigated, and the results are provided in FIG. 15. The results demonstrate that the dynamic binding capacity reached a plateau at 4 minutes, with a value of 9.3 mg/mL. Subsequent pilot scale work using HCCF demonstrated the potential to increase load density up to 19 mg/mL, such as about 10, 11, 12, 13, 14, 15, 16, 17, or about 18 mg/mL. As such, in some embodiments of the subject methods, a CH1-XL chromatography step comprises a load density that ranges from about 9 to about 19 mg/mL, such as about 10, 11, 12, 13, 14, 15, 16, 17 or about 18 mg/mL.

### Elution

Elution, as used herein, refers to the removal or dissociation of the product, e.g., a multispecific antibody, from the chromatography material. Elution buffer is the buffer used to elute the multispecific antibody from a chromatography material. In some embodiments, the elution buffer may comprise citrate, acetate, acetic acid, 4-Morpholineethanesulfonate (MES), citrate-phosphate, succinate, and the like. In certain embodiments, the elution buffer used to elute multispecific antibodies from an affinity chromatography column comprising Protein A comprises citrate in a concentration that ranges from about 5 mM to about 50 mM, such as about 10, 15, 20, 25, 30, 35, 40, or about 45 mM. In some embodiments, the concentration of citrate in an elution buffer ranges from about 20 mM to about 30 mM. In some embodiments, the elution buffer comprises citrate in a concentration of about 25 mM. In certain embodiments, the elution buffer used to elute multispecific antibodies from an affinity chromatography column comprising a domain-specific chromatography resin which binds to a CH1 domain of an IgG antibody comprises acetic acid in a concentration that ranges from about 5 mM up to about 60 mM, such as about 10, 15, 20, 25, 30, 35, 40, 45, 50, or 55 mM. In some embodiments, the concentration of acetic acid in an elution buffer ranges from about 45 mM to about 55 mM. In some embodiments, the elution buffer comprises acetic acid in a concentration of about 50 mM.

It was found that the pH of the elution buffer affects multispecific antibody aggregation. Thus, in one embodiment, the elution buffer used to elute multispecific antibodies from an affinity chromatography column comprising Protein A has a pH that ranges from about 3.2 to about 4.2, such as 3.2, 3.3, 3.4, 3.5, 3.6, 3.7, 3.8, 3.9, 4.0, 4.1, or 4.2. In certain embodiments, the elution buffer used to elute multispecific antibodies from an affinity chromatography column comprising Protein A has a pH that ranges from about 3.4 to about 3.8. In some embodiments, the elution buffer used to elute multispecific antibodies from an affinity chromatography column comprising a domain-specific chromatography resin which binds to a CH1 domain of an IgG antibody has a pH that ranges from about 3.4 to about 4.4, such as 3.4, 3.5, 3.6, 3.7, 3.8, 3.9, 4.0, 4.1, 4.2, 4.3, or 4.4. In some embodiments, the elution buffer used to elute multispecific antibodies from an affinity chromatography column comprising a domain-specific chromatography resin which binds to a CH1 domain of an IgG antibody has a pH that ranges from 3.8 to about 4.2. In some embodiments, the elution buffer used to elute multispecific antibodies from an affinity chromatography column comprising a domain-specific chromatography resin which binds to a CH1 domain of an IgG antibody has a pH of about 4.0.

### Anti-Aggregation Composition

Initial studies revealed that BsAb purification with Protein A resin resulted in high molecular weight aggregates when the Protein A resin was eluted at low pH. It was discovered that by supplementing the elution buffer with additives, the amount of BsAb aggregates was reduced. Thus, in certain preferred embodiments, an anti-aggregation composition is added to the elution buffer before elution of the multispecific antibody. In some embodiments, an anti-aggregation composition comprises one or more polyols. Non-limiting examples of polyols include mannitol, glycerol, sucrose, trehalose, and sorbitol. In some embodiments, the elution buffer comprises an anti-aggregation composition comprising one or more polyols. In certain embodiments, the one or more polyols are selected from the group consisting of: mannitol, glycerol, sucrose, trehalose, and combinations thereof. In some embodiments, the one or more polyols have a concentration that ranges from about 5% to about 25% w/v, such as 5%, 10%, 15%, or 20% w/v. In other embodiments, the one or more polyols comprise glycerol, having a concentration that ranges from about 5% to about 15% w/v. In one embodiment, the elution buffer comprises glycerol at a concentration of about 10% w/v. In other embodiments, the one or more polyols comprise sucrose, having a concentration that ranges from about 5% to about 15% w/v. In some embodiments, the elution buffer comprises sucrose at a concentration of about 10% w/v. In certain embodiments, the elution buffer comprises about 10% glycerol and about 10% sucrose w/v. Methods in accordance with embodiments of the invention include Protein A chromatography using an elution buffer comprising any combination of the additives described herein, at any pH described herein. Further methods in accordance with embodiments of the invention include affinity chromatography comprising a domain-specific chromatography resin that binds to a CH1 domain of an IgG antibody using an elution buffer comprising any combination of the additives described herein, at any pH described herein. In certain embodiments, the affinity chromatography comprising a domain-specific chromatography resin that binds to a CH1 domain of an IgG antibody is a CaptureSelect™ resin. In some embodiments, the CaptureSelect™ resin is CaptureSelect™ CH1-XL.

### Downstream Purification Processes

In certain embodiments, the eluate from the affinity chromatography is subject to one or more additional purification steps. For example, in certain embodiments, the eluate from the affinity chromatography step is subsequently applied to, e.g., an anion-exchange chromatography procedure and/or a cation exchange chromatography procedure.

Anion exchange chromatography material is a solid phase that is positively charged and has free anions for exchange with anions in an aqueous solution (such as a composition comprising a multispecific antibody and an impurity) that is passed over or through the solid phase. In some embodiments of any of the methods described herein, the anion exchange material may be a membrane, a monolith, or resin. In one embodiment, the anion exchange material is a resin. In some embodiments, the anion exchange material may comprise a primary amine, a secondary amine, a tertiary amine or a quaternary ammonium ion functional group, a polyamine functional group, or a diethylaminoaethyl functional group. Examples of anion exchange materials are known in the art and include, but are not limited to Poros® HQ 50, Poros® PI 50, Poros® D, Mustang® Q, Q Sepharose® Fast Flow (QSFF), Accell™ Plus Quaternary Methyl Amine (QMA) resin, Sartobind STIC®, and DEAE-Sepharose®. In some embodiments, the anion exchange chromatography is performed in “bind and elute” mode. In some embodiments, the anion exchange chromatography is performed in “flow through” mode. In some embodiments, the anion exchange chromatography material is provided in the form of a column. In some embodiments, the anion exchange chromatography material comprises a membrane.

Cation exchange chromatography material is a solid phase that is negatively charged and has free anions for exchange with cations in an aqueous solution (such as a composition comprising a multispecific antibody and an impurity) that is passed over or through the solid phase. In some embodiments of any of the methods described herein, the cation exchange material may be a membrane, a monolith, or resin. In some embodiments, the cation exchange material is a resin. The cation exchange material may comprise a carboxylic acid functional group or a sulfonic acid functional group such as, but not limited to, sulfonate, carboxylic, carboxymethyl sulfonic acid, sulfoisobutyl, sulfoethyl, carboxyl, sulphopropyl, sulphonyl, sulphoxyethyl, or orthophosphate. In some embodiments of the above, the cation exchange chromatography material is a cation exchange chromatography column. In some embodiments of the above, the cation exchange chromatography material is a cation exchange chromatography membrane. Examples of cation exchange materials are known in the art include, but are not limited to Mustang® S, Sartobind® S, S03 Monolith (such as, e.g., CIM®, CIMmultus® and CIMac® S03), S Ceramic HyperD®, Poros® XS, Poros® HS 50, Poros® HS 20, sulphopropyl-Sepharose® Fast Flow (SPSFF), SP-Sepharose® XL (SPXL), CM Sepharose® Fast Flow, Capto™ S, Fractogel® EMD Se Hicap, Fractogel® EMD S03, or Fractogel® EMD COO. In some embodiments, the cation exchange chromatography is performed in “bind and elute” mode. In some embodiments, the cation exchange chromatography is performed in “flow through” mode. In some embodiments of the above, the cation exchange chromatography material is in a column. In some embodiments of the above, the cation exchange chromatography material comprises a membrane.

In some embodiments, the eluate from the anion-exchange or cation-exchange chromatography is subject to mixed mode chromatography.

Mixed mode chromatography is chromatography that utilizes a mixed mode media, such as, but not limited to Capto Adhere™ available from GE Healthcare. Such a media comprises a mixed mode chromatography ligand. In certain embodiments, such a ligand refers to a ligand that is capable of providing at least two different, but co-operative, sites which interact with the substance to be bound. One of these sites gives an attractive type of charge-charge interaction between the ligand and the substance of interest. The other site typically gives electron acceptor-donor interaction and/or hydrophobic and/or hydrophilic interactions. Electron donor-acceptor interactions include interactions such as hydrogen-bonding, π-π, cation-π, charge transfer, dipole-dipole, induced dipole, etc.

In certain embodiments, the mixed mode (MM) chromatography media is comprised of mixed mode ligands coupled to an organic or inorganic support, sometimes denoted a base matrix, directly or via a spacer. The support may be in the form of particles, such as essentially spherical particles, a monolith, filter, membrane, surface, capillaries, etc. In certain embodiments, the support is prepared from a native polymer, such as cross-linked carbohydrate material, such as agarose, agar, cellulose, dextran, chitosan, konjac, carrageenan, gellan, alginate etc. To obtain high adsorption capacities, the support can be porous, and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964). Alternatively, the support can be prepared from a synthetic polymer, such as cross-linked synthetic polymers, e.g. styrene or styrene derivatives, divinylbenzene, acryl amides, acrylate esters, methacrylate esters, vinyl esters, vinyl amides etc. Such synthetic polymers can be produced according to standard methods, see e.g. “Styrene based polymer supports developed by suspension polymerization” (R Arshady: Chimica e L'Industria 70(9), 70-75 (1988)). Porous native or synthetic polymer supports are also available from commercial sources, such as GE Healthcare (Uppsala, Sweden).

In certain embodiments, the mixed-mode resin comprises a negatively charged part and a hydrophobic part. In one embodiment, the negatively charged part is an anionic carboxylate group or anionic sulfo group for cation exchange. Examples of such supports include, but are not limited to, Capto Adhere® (GE Healthcare). Capto Adhere® is a strong anion exchanger with multimodal functionality which confers different selectivity to the resin compared to traditional anion exchangers. The Capto Adhere® ligand (N-Benzyl-N-methyl ethanolamine) exhibits multiple modes of protein-interactive chemistries, including ionic interaction, hydrogen bonding and hydrophobic interaction. The multimodal functionality of the resin confers it with an ability to remove antibody dimers and aggregates, leached protein A, host cell proteins (HCP), antibody/HCP complexes, process residuals and viruses. The resin may be used in flow-through mode in the context of a production scale polishing step employing operational parameters designed to have the multispecific antibody pass directly through the column while the contaminants are adsorbed.

In certain embodiments, the purified multispecific binding compound is subjected to a viral filtration step. Viral filtration is a dedicated viral reduction step in the entire purification process. This step is usually performed post chromatographic polishing steps. Viral reduction can be achieved via the use of suitable filters including, but not limited to, Planova 20N™, 50 N or BioEx from Asahi Kasei Pharma, Viresolve™ filters from EMD Millipore, ViroSart CPV from Sartorius, Sartorius filters, Zeta Plus VR™ filters from CUNO, or Ultipor DV20 or DV50™ filter from Pall Corporation. It will be apparent to one of ordinary skill in the art to select a suitable filter to obtain desired filtration performance.

Certain embodiments of the present invention employ ultrafiltration (UF) and/or diafiltration (DF) steps to further purify and concentrate the antibody sample. Typically, this is carried out following one or more of the purification steps described herein. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). A preferred filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size that allow transfer of protein with average size of 50 kDa (for example) or smaller. By employing filters having such small pore size, the volume of the sample can be reduced through permeation of the sample buffer through the filter while antibodies are retained behind the filter.

Diafiltration is a method of using ultrafilters to remove and exchange salts, sugars, and non-aqueous solvents, to separate free from bound species, to remove low molecular-weight material, and/or to cause the rapid change of ionic and/or pH environments. Microsolutes are removed most efficiently by adding solvent to the solution being ultrafiltered at a rate approximately equal to the ultratfiltration rate. This washes microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the multispecific binding agents.

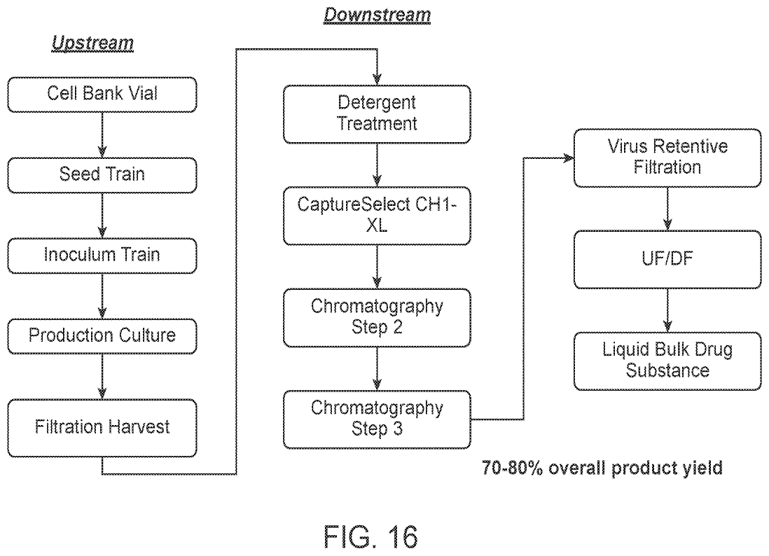

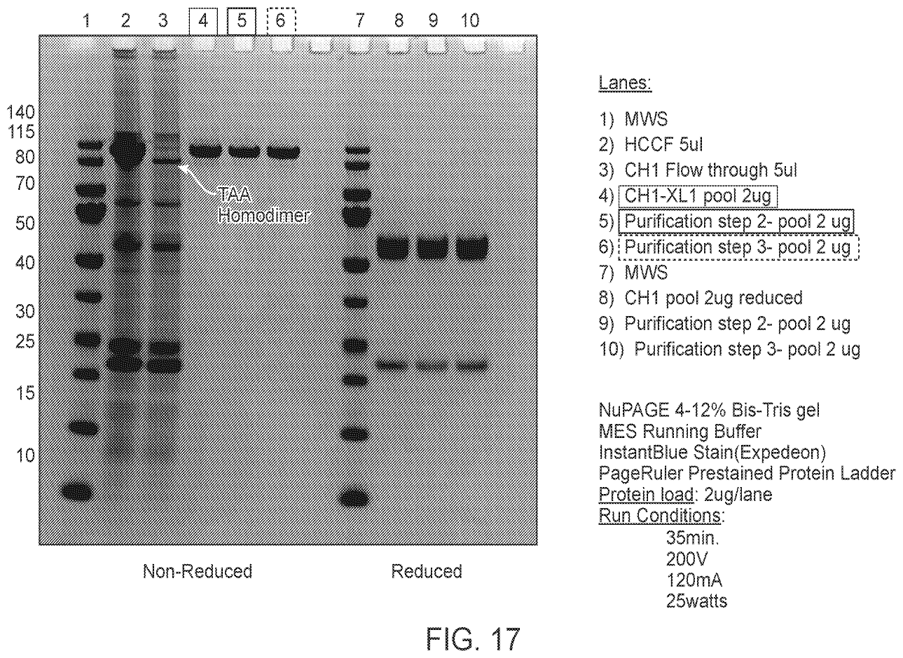

A schematic flow diagram of a manufacturing process that can be used to produce a BsAb in accordance with embodiments of the invention is provided in FIG. 16. The flow diagram shows representative upstream and downstream unit operations. An analysis of the BsAb species found at each stage of the purification process is provided in FIG. 17. The results demonstrate removal of the TAA homodimer species after the CH1-XL chromatography step. Overall yield for a manufacturing processes in accordance with embodiments of the invention ranged from about 70% to about 90%, such as about 75%, 80%, or about 85%. Thus, in some embodiments, the purification methods of the present invention result in an overall yield of multispecific antibody product of at least about 70%, about 75%, about 80%, about 85%, about 90%, about 95%.

### Pharmaceutical Compositions

It is another aspect of the present invention to provide pharmaceutical compositions comprising one or more multispecific antibodies purified by the methods of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

Pharmaceutical composition of the multispecific antibodies purified in accordance with the present invention are prepared for storage by mixing proteins having the desired degree of purity with optional pharmaceutically acceptable carriers, excipients or stabilizers (see, e.g., Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen. The multispecific antibodies purified according to the methods described herein can be administered by intravenous injection or infusion or subcutaneously. For injection administration, the multispecific antibodies purified according to the methods described herein can be formulated in aqueous solutions, preferably in physiologically-compatible buffers to reduce discomfort at the site of injection. The solution can contain carriers, excipients, or stabilizers as discussed above. Alternatively, multispecific antibodies can be in lyophilized form for constitution with a suitable vehicle, e.g., sterile pyrogen-free water, before use.

### Articles of Manufacture

The multispecific antibodies purified by the methods described herein and/or formulations comprising the polypeptides purified by the methods described herein may be contained within an article of manufacture. The articles of manufacture, or “kits”, containing one or more multispecific antibodies purified according to the methods of the invention are useful for the treatment of the diseases and disorders described herein. In one embodiment, a kit comprises a container comprising a multispecific antibody, e.g., a bispecific anti-CD3 antibody, purified as described herein. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister packs, etc. The container may be formed from a variety of materials such as glass or plastic. The container may hold one or more multispecific antibodies as described herein, or a formulation thereof, e.g., a combination formulation of two or more multispecific antibodies, which is effective for treating a condition and may have a sterile access port (for example, the container may be an intravenous solution bag or a vial having a stopper pierceable by a hypodermic injection needle). The label or package insert indicates that the composition is used for treating the condition of choice, such as a cancer or an immunological disorder. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of one or more multispecific antibodies and, if present, a combination formulation thereof. For example, if the kit comprises a first pharmaceutical composition comprising a first multispecific antibody and a second pharmaceutical composition comprising a second multispecific antibody, the kit may further comprise directions for the simultaneous, sequential or separate administration of the first and second pharmaceutical compositions to a patient in need thereof. Where a kit comprises two or more compositions, the kit may comprise a container for containing the separate compositions, such as a divided bottle or a divided foil packet, however, the separate compositions may also be contained within a single, undivided container. A kit can comprise directions for the administration of the separate components, or for the administration a combined formulation thereof.

### Methods of Use

In certain aspects, the present invention provides methods for purifying a multispecific antibody from a mixture using an affinity chromatography procedure, comprising contacting a first affinity chromatography column with the mixture, immobilizing the multispecific antibody on the first affinity chromatography column, contacting the first affinity chromatography column with an elution buffer, wherein the elution buffer comprises an anti-aggregation composition, and eluting the multispecific antibody from the first affinity chromatography column to purify the multispecific antibody from the mixture.

In certain embodiments, the anti-aggregation composition comprises one or more polyols. In some embodiments, the one or more polyols are selected from the group consisting of: mannitol, glycerol, sucrose, trehalose, and combinations thereof. In certain embodiments, the one or more polyols have a concentration that ranges from about 5% to about 25% w/v. In some embodiments, the one or more polyols comprise glycerol, having a concentration that ranges from about 5% to about 15% w/v. In certain embodiments, glycerol has a concentration of about 10% w/v. In other embodiments, the one or more polyols comprise sucrose, having a concentration that ranges from about 5% to about 15% w/v. In some embodiments, sucrose has a concentration of about 10% w/v. In other embodiments, the elution buffer comprises about 10% glycerol and about 10% sucrose w/v.

In certain embodiments, the affinity chromatography column comprises a Protein A chromatography resin. In some embodiments, the elution buffer is selected from the group consisting of: citrate, acetate, acetic acid, 4-Morpholineethanesulfonate (MES), citrate-phosphate, succinate, and combinations thereof. In some embodiments, the elution buffer comprises citrate in a concentration that ranges from about 20 mM to about 30 mM. In other embodiments, the elution buffer comprises citrate in a concentration of about 25 mM. In certain of these embodiments, the elution buffer has a pH that ranges from about 3.2 to about 4.2. In other embodiments, the elution buffer has a pH that ranges from about 3.4 to about 3.8. In certain embodiments, the elution buffer has a pH of about 3.6. In some embodiments, the elution buffer comprises about 25 mM citrate, about 10% glycerol, and about 10% sucrose, and wherein the elution buffer has a pH of about 3.6.

In other embodiments, the affinity chromatography comprises a domain-specific chromatography resin that binds to a CH1 domain of an IgG antibody. In certain of these embodiments, the elution buffer comprises a buffer selected from the group consisting of: citrate, acetate, acetic acid, 4-Morpholineethanesulfonate (MES), citrate-phosphate, succinate, and combinations thereof. In some embodiments, the elution buffer comprises acetic acid in a concentration that ranges from about 45 mM to about 55 mM. In certain embodiments, the elution buffer comprises acetic acid in a concentration of about 50 mM. In some embodiments, the elution buffer has a pH that ranges from about 3.4 to about 4.4. In yet other embodiments, the elution buffer has a pH that ranges from about 3.8 to about 4.2. In some embodiments, the elution buffer has a pH of about 4.0. In certain embodiments, the elution buffer comprises about 50 mM acetic acid, about 10% glycerol, and about 10% sucrose, and wherein the elution buffer has a pH of about 4.0.

In other aspects, the invention provides methods of reducing aggregation of a multispecific antibody in an elution pool from an affinity chromatography procedure comprising contacting a protein A affinity chromatography column with a mixture comprising the multispecific antibody, immobilizing the multispecific antibody on the protein A affinity chromatography column, contacting the protein A affinity chromatography column with an elution buffer, wherein the elution buffer comprises 25 mM citrate, 10% glycerol, and 10% sucrose w/v, and wherein the elution buffer has a pH of 3.6, and eluting the multispecific antibody from the protein A affinity chromatography column to purify the multispecific antibody from the mixture.

In yet other aspects, the invention provides methods of reducing aggregation of a multispecific antibody in an elution pool from an affinity chromatography procedure comprising contacting an affinity chromatography column comprising a domain-specific chromatography resin which binds to a CH1 domain of an IgG antibody with a mixture comprising the multispecific antibody, immobilizing the multispecific antibody on the affinity chromatography column comprising the domain-specific chromatography resin, contacting the affinity chromatography column comprising the domain-specific chromatography resin with an elution buffer, wherein the elution buffer comprises 50 mM acetic acid, 10% glycerol and 10% sucrose, and wherein the elution buffer has a pH of 4.0, eluting the multispecific antibody from the affinity chromatography column comprising the domain-specific chromatography resin to purify the multispecific antibody from the mixture.

In all aspects, the multispecific antibody may comprise a first and a second binding unit. In some embodiments, one of the binding units comprises a heavy chain variable region of a heavy chain-only antibody. In some embodiments, both the first and the second binding units comprise a heavy chain variable region of a heavy chain-only antibody. In other embodiments, one of the binding units comprises a heavy chain variable region of an antibody and a light chain variable region of an antibody. In other embodiments, both the first and the second binding units comprise a heavy chain variable region of an antibody and a light chain variable region of an antibody. In still other embodiments, the first binding unit comprises a heavy chain variable region of a heavy chain-only antibody and the second binding unit comprises a heavy chain variable region of an antibody and a light chain variable region of an antibody.

In some embodiments, the first binding unit has binding affinity to a tumor-associated antigen. In certain embodiments, the second binding unit has binding affinity to an effector cell. In some embodiments, the effector cell is a T cell. In some embodiments, the second binding unit has binding affinity to a CD3 protein on the T cell.

In all aspects of the invention, a multispecific antibody can be a bispecific antibody.

The multispecific antibody purified as disclosed herein or the composition comprising the multispecific antibody and a pharmaceutically acceptable carrier is then used for various diagnostic, therapeutic or other uses known for such multispecific antibody and compositions. For example, the multispecific antibody may be used to treat a disorder in a mammal by administering a therapeutically effective amount of the multispecific antibody to the mammal.

The invention now being fully described, it will be apparent to one of ordinary skill in the art that various changes and modifications can be made without departing from the spirit or scope of the invention.

### EXAMPLES

### Example 1: Purification of an Anti-CD3-BCMA Bispecific Antibody

A BsAb CD3-BCMA, depicted in FIG. 2, was purified as follows. BsAb CD3-BCMA is a bispecific antibody and is structurally a trimer, in which one arm (e.g., a CD3-binding arm) contains both fully human heavy and κ light chains, while the other arm (e.g., a BCMA arm), derived from UniRat™ technology, consists of a human heavy chain (with one or more VH domains fused directly into a CH domain (comprising, e.g., hinge-CH2-CH3, and lacking a CH1 domain). The variable domain sequences comprising BsAb CD3-BCMA are shown in Table 1 below. Specifically, BsAb CD3-BCMA is a fully human IgG4 bispecific monoclonal antibody having two heavy chains (HC-1 and HC-2 and one kappa light chain (κLC) and is acid labile. Correct pairing of heavy chains is achieved through knobs-into-holes technology. The CD3 arm comprises HC-1 and κLC and binds the T-cell receptor CD3. The TAA, or BCMA, arm comprises HC-2 only and consists of two identical VH domains recognizing BCMA. The TAA arm is bivalent for increased avidity (<1 nM) and is derived from UniRat™ technology. Due to the unique structure of this BsAb, only the heterodimeric product contains a CH1 domain of human heavy chain (part of the CD3-binding arm).

TABLE 1
BsAb CD3-BCMA variable domain sequences.

| | |
|---|---|
| Anti-CD3 Heavy chain | Glu Val Gln Leu Val Glu Ser Gly Gly Gly Leu Val Gln Pro Gly Arg |
| variable domain | Ser Leu Arg Leu Ser Cys Ala Ala Ser Gly Phe Thr Phe Asp Asp Tyr |
| | Ala Met His Trp Val Arg Gln Ala Pro Gly Lys Gly Leu Glu Trp Val |
| | Ser Gly Ile Ser Trp Asn Ser Gly Ser Ile Gly Tyr Ala Asp Ser Val |
| | Lys Gly Arg Phe Thr Ile Ser Arg Asp Asn Ala Lys Asn Ser Leu Tyr |
| | Leu Gln Met Asn Ser Leu Arg Ala Glu Asp Thr Ala Leu Tyr Tyr Cys |
| | Ala Lys Asp Ser Arg Gly Tyr Gly Asp Tyr Arg Leu Gly Gly Ala Tyr |
| | Trp Gly Gln Gly Thr Leu Val Thr Val Ser Ser |
| Anti-CD3 Light chain | Glu Ile Val Met Thr Gln Ser Pro Ala Thr Leu Ser Val Ser Pro Gly |
| variable domain | Glu Arg Ala Thr Leu Ser Cys Arg Ala Ser Gln Ser Val Ser Ser Asn |
| | Leu Ala Trp Tyr Gln Gln Lys Pro Gly Gln Ala Pro Arg Leu Leu Ile |
| | Tyr Gly Ala Ser Thr Arg Ala Thr Gly Ile Pro Ala Arg Phe Ser Gly |
| | Ser Gly Ser Gly Thr Glu Phe Thr Leu Thr Ile Ser Ser Leu Gln Ser |
| | Glu Asp Phe Ala Val Tyr Tyr Cys Gln Gln Tyr Asn Asn Trp Pro Trp |
| | Thr Phe Gly Gln Gly Thr Lys Val Glu Ile Lys |
| Anti-BCMA heavy chain | Glu Val Gln Leu Leu Glu Ser Gly Gly Gly Leu Val Gln Pro Gly Gly |
| variable domain | Ser Leu Arg Leu Ser Cys Ala Ala Ser Gly Phe Thr Val Ser Ser Tyr |
| | Gly Met Ser Trp Val Arg Gln Ala Pro Gly Lys Gly Pro Glu Trp Val |
| | Ser Gly Ile Arg Gly Ser Asp Gly Ser Thr Tyr Tyr Ala Asp Ser Val |
| | Lys Gly Arg Phe Thr Ile Ser Arg Asp Asn Ser Lys Asn Thr Leu Tyr |
| | Leu Gln Met Asn Ser Leu Arg Ala Glu Asp Thr Ala Val Tyr Tyr Cys |
| | Ala Lys Gln Gly Glu Asn Asp Gly Pro Phe Asp His Arg Gly Gln Gly |
| | Thr Leu Val Thr Val Ser Ser Gly Gly Gly Gly Ser Glu Val Gln Leu |
| | Leu Glu Ser Gly Gly Gly Leu Val Gln Pro Gly Gly Ser Leu Arg Leu |
| | Ser Cys Ala Ala Ser Gly Phe Thr Val Ser Ser Tyr Gly Met Ser Trp |
| | Val Arg Gln Ala Pro Gly Lys Gly Pro Glu Trp Val Ser Gly Ile Arg |
| | Gly Ser Asp Gly Ser Thr Tyr Tyr Ala Asp Ser Val Lys Gly Arg Phe |
| | Thr Ile Ser Arg Asp Asn Ser Lys Asn Thr Leu Tyr Leu Gln Met Asn |
| | Ser Leu Arg Ala Glu Asp Thr Ala Val Tyr Tyr Cys Ala Lys Gln Gly |
| | Glu Asn Asp Gly Pro Phe Asp His Arg Gly Gln Gly Thr Leu Val Thr |
| | Val Ser Ser |

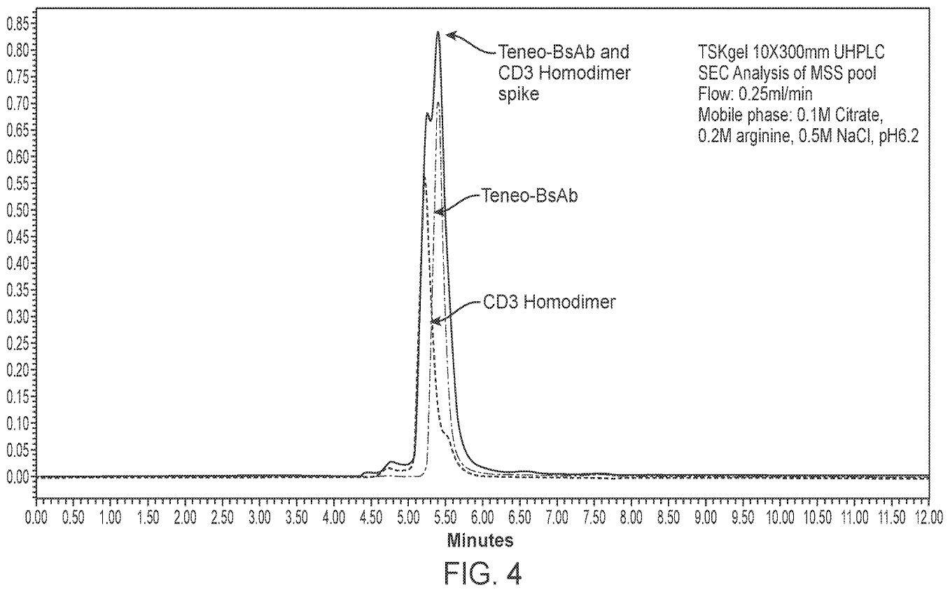

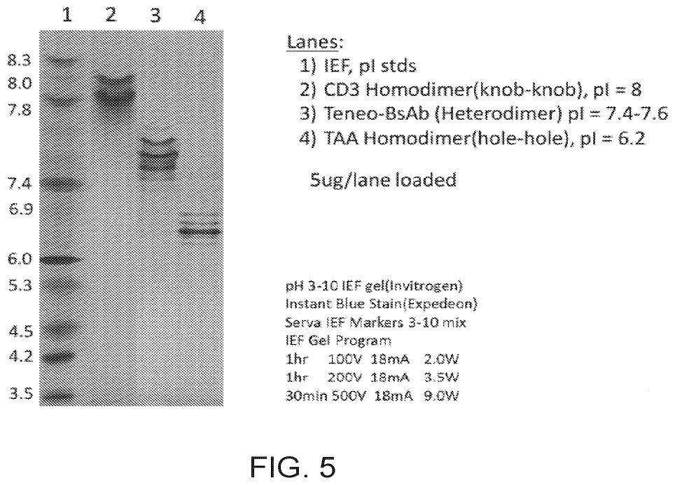

Size exclusion chromatography (SEC) analysis of the various species depicted in FIG. 3 demonstrates that the BsAb heterodimer is similar in size to the HC/LC homodimer species (e.g., the CD3 homodimer species). The SEC parameters were: TSKgel 10×300 mm UHPLC SEC Analysis of MSS pool with a flow of 0.25 ml/min; mobile phase: 0.1M citrate, 0.2M arginine, 0.5M NaCl, pH 6.2. These results are shown in FIG. 4. Moreover, an analysis of the isoelectric points (pIs) of these species reveals that the heterodimer and homodimers have distinct pIs. These results are shown in FIG. 5. Lane 1 is the isoelectric focusing (IEF) pI standards. Lane 2 is the CD3 homodimer (knob-knob), pI=8. Lane 3 is the CD3/BCMA bispecific IgG, pI=7.4-7.6. Lane 4 is the BCMA homodimer (hole-hole), pI=6.2. 5 pig/lane was loaded. IEF parameters were as follows: pH 3-10 IEF gel (Invitrogen); Instant Blue Stain (Expedeon); Serva IEF markers 3-10 mix); IEF Gel Program 1 hr at 200V, 18 mA, 2.0 W; 1 hr at 200V, 18 mA, 3.5 W; 30 min at 500V, 18 mA, 9.0 W.

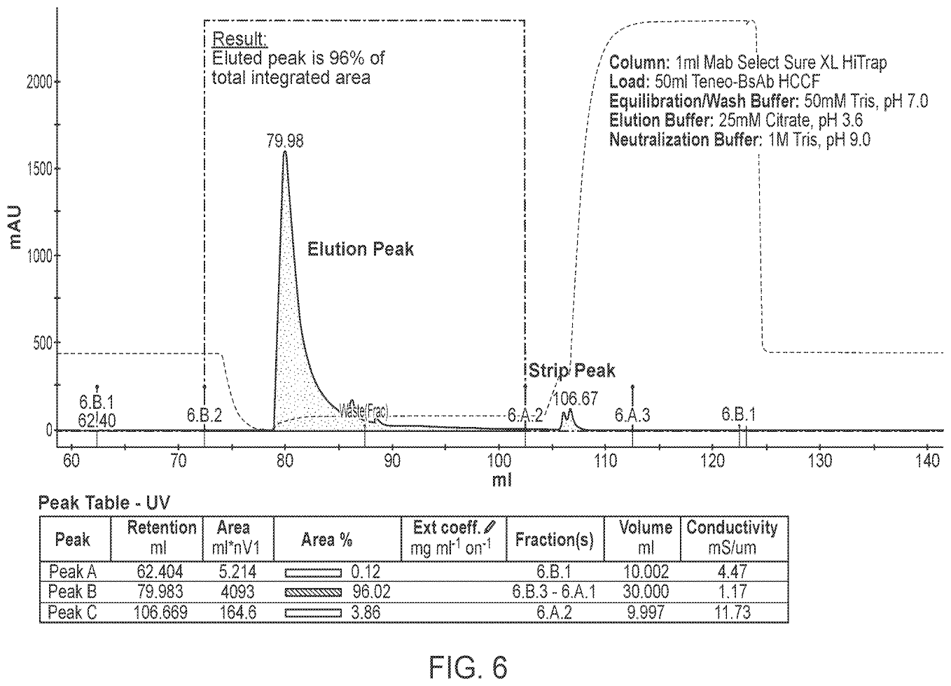

Initial studies revealed that BsAb purification with Protein A resin was efficient as shown in FIG. 6, which shows that the eluted peak is 90% of the total integrated area. The Protein A chromatography parameters were as followed: Column: 1 ml MabSelect™ SuRe™ LX HiTrap®, GE Healthcare Life Sciences; Load: 50 mL Teneo-BsAb HCCF; Equilibration/Wash Buffer: 50 mM Tris, pH 7.0; Elution Buffer: 25 mM Citrate, pH 3.6; Neutralization Buffer: 1M Tris, pH 9.0.

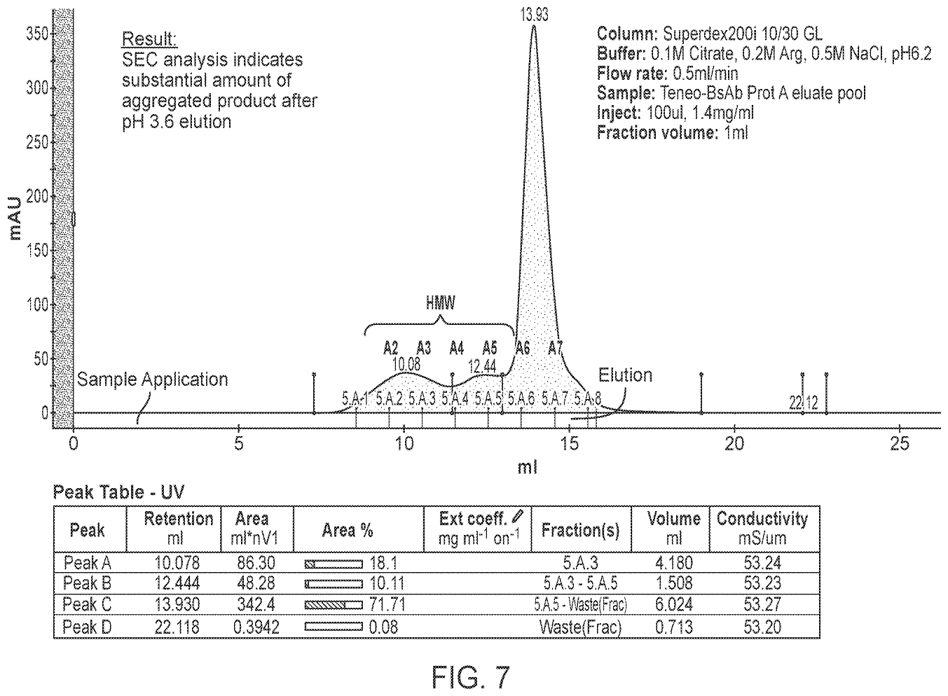

Initial studies also revealed that BsAb purification with Protein A resin resulted in high molecular weight (HMW) aggregates that were undesirable (FIG. 7). SEC analysis indicated substantial amounts of aggregated produce after pH 3.6 elution. SEC parameters were as follows: Column: Superdex200i 10/30 GL; Buffer: 0.1M Citrate, 0.2M Arg, 0.5M NaCl, pH 6.2; Flow rate: 0.5 ml/min; Sample: TeneoBsAb Prot A eluate pool; Inject: 100 μl, 1.4 mg/mL; Fraction volume: 1 mL.

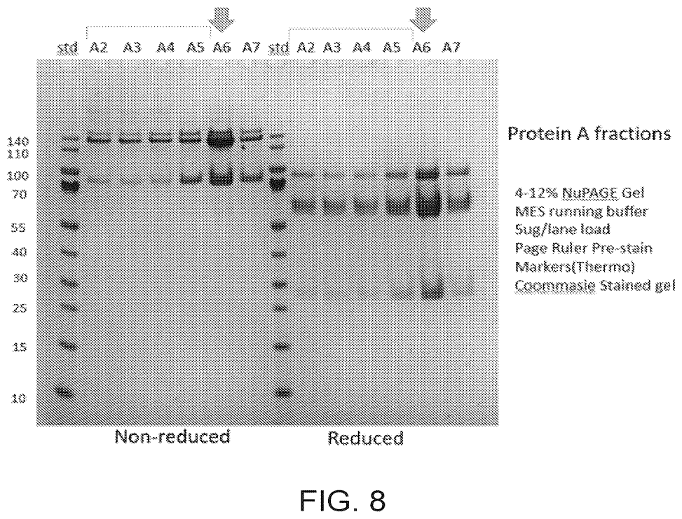

SDS-PAGE analysis confirmed that the HMW fractions included the BsAb product (FIG. 8). Lanes A2-A5: aggregates; Lanes A6: monomer. SDS-PAGE parameters were: 4-12% NuPAGE gel; MES running buffer; 5 μg/lane load; Page Ruler Pre-stain; Markers (ThermoFisher Scientific); Coommasie stained gel.

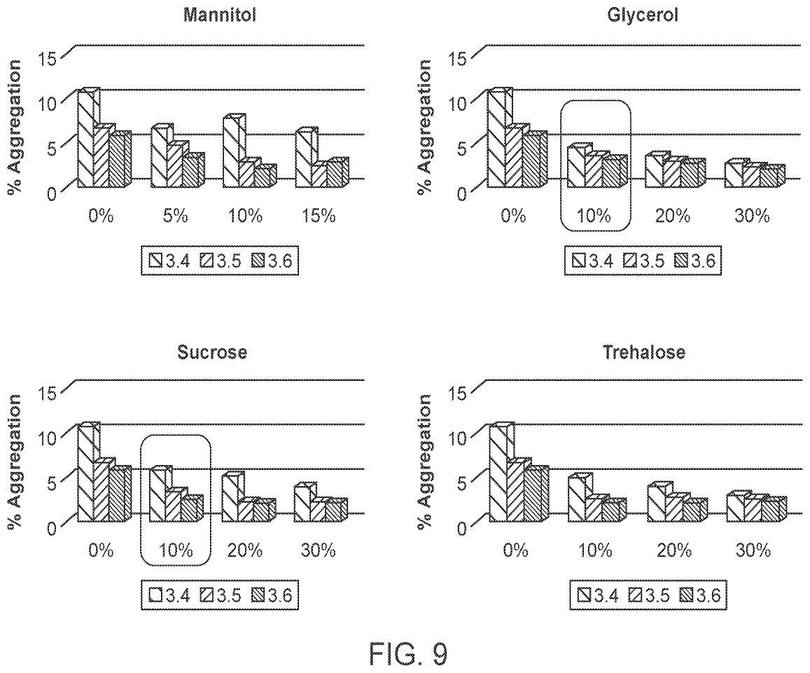

Accordingly, additives were investigated to determine whether Protein A purification could be improved by reducing the amount of BsAb aggregates. To this end, the Protein A elution buffer was supplemented with varying amounts of different polyols. Three factors were tested in a design of experiments (DOE) matrix: type of polyol, percentage of polyol, and pH of the elution buffer. The types of polyols investigated were mannitol, glycerol, sucrose and trehalose. The percentages tested ranged from 0% to 30%, such as 5%, 10%, 15%, 20% or 25%. The pH of the elution buffer was 3.4, 3.5, or 3.6. The results of the various combinations tested are shown in FIG. 9. Methods in accordance with embodiments of the invention include Protein A chromatography using an elution buffer comprising any combination of the additives described above, at any pH described above.

The results of these tests demonstrated that additives to the Protein A elution buffer can reduce aggregation of the BsAb product. Lowest levels of aggregation were observed with an elution buffer comprising 10% glycerol and 10% sucrose. This elution buffer composition was the most optimal of the compositions tested. As such, in one preferred embodiment, a method for purifying a BsAb comprises a Protein A chromatography step, wherein the Protein A elution buffer comprises 10% glycerol and 10% sucrose.

In spite of the reduced levels of aggregation observed from adding the above-described additives to the Protein A elution buffer, co-purification of unwanted homodimer species (e.g., TAA homodimer species) was still observed. In addition, the acid lability of the BsAb precludes use of low pH viral inactivation unit operations.

Two criteria thus guided the search for a chromatography resin that could be used as an alternative to Protein A: (a) the ability to elute product under mild (less acidic) conditions, and (b) selectivity for the heterodimeric product over the heavy chain homodimer as a process impurity. CaptureSelect™ CH1-XL, commercially available from ThermoFisher, is an affinity resin that binds specifically to the CH1 domain on the heavy chain of human IgG with the benefits of a robust and high quality affinity matrix provided by a 13 kDa llama heavy chain antibody fragment.

The general properties of the CH1-XL resin are that it comprises an Ig heavy chain CH1-specific nanobody ligand; it recognized all four subclasses of IgG (i.e., IgG1, IgG2, IgG3 and IgG4); it is ligand immobilized on agarose having a size of 65 μm; it has a binding capacity of less than 20 mg/mL of IgG; it can be used under flow rate conditions of 5-200 cm/hr; it is stable to base (25-50 mM NaOH) for sanitization; and is commercially available. For purposes of purifying BsAb, the CH1-XL resin binds to bispecific heterodimer comprising a CH1 domain, but does not bind to the heavy chain homodimer species (e.g., the TAA homodimer). As shown in FIG. 10, only the active species includes a CH1 domain. In addition, the CH1-XL resin can be used under less stringent acidic elution conditions (pH 4).

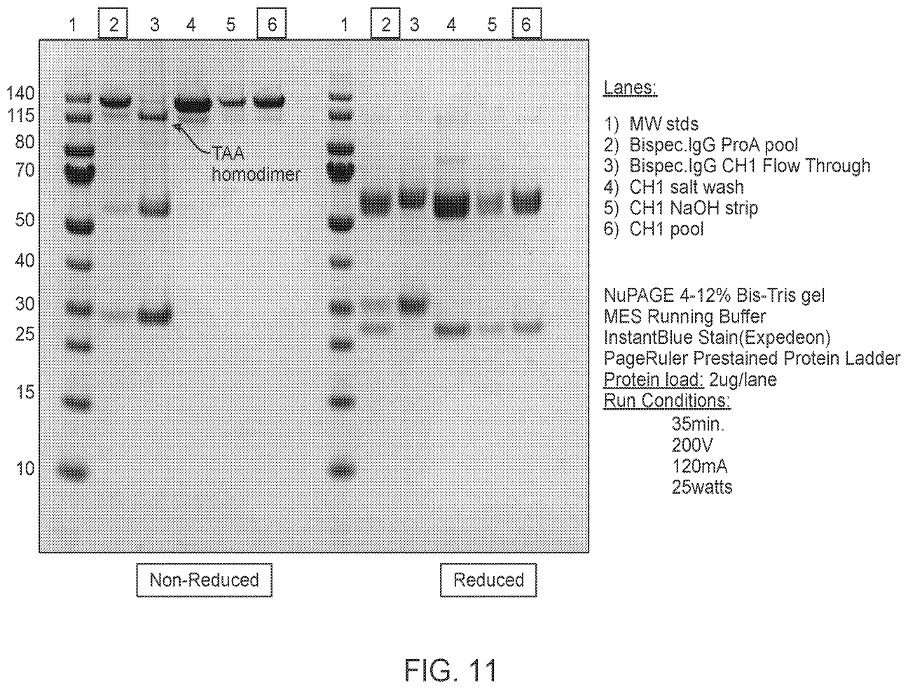

Investigation of the CH1-XL resin demonstrated that the heavy chain homodimer was present in the CH1-XL flow through, indicating that, as anticipated, this homodimer does not bind to the resin (FIG. 11). As shown in FIG. 11, Lane 1 is the molecular weight standards (5 μl). Lane 2 is the bispecific IgG Protein A pool (2 μg). Lane 3 is the bispecific IgG CH1 flowthrough (2 μg). Lane 4 is the CH1 salt wash (2 μg). Lane 5 is the CH1 NaOH strip (2 μg). Lane 6 is the CH1 pool (2 μg). SDS-PAGE parameters were: Protein load: 2 μg/lane; NuPAGE 4-12% Bis-Tris gel; MES running buffer; InstantBlue stain (Expedeon); PageRuler prestained protein ladder; Run conditions: 35 min., 200V, 120 mA, 25 watts.

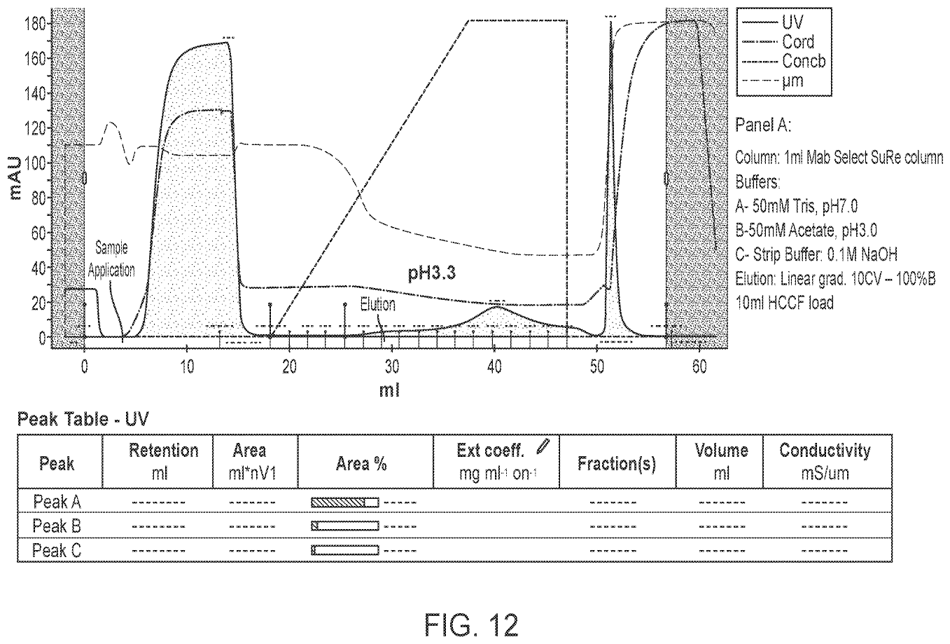

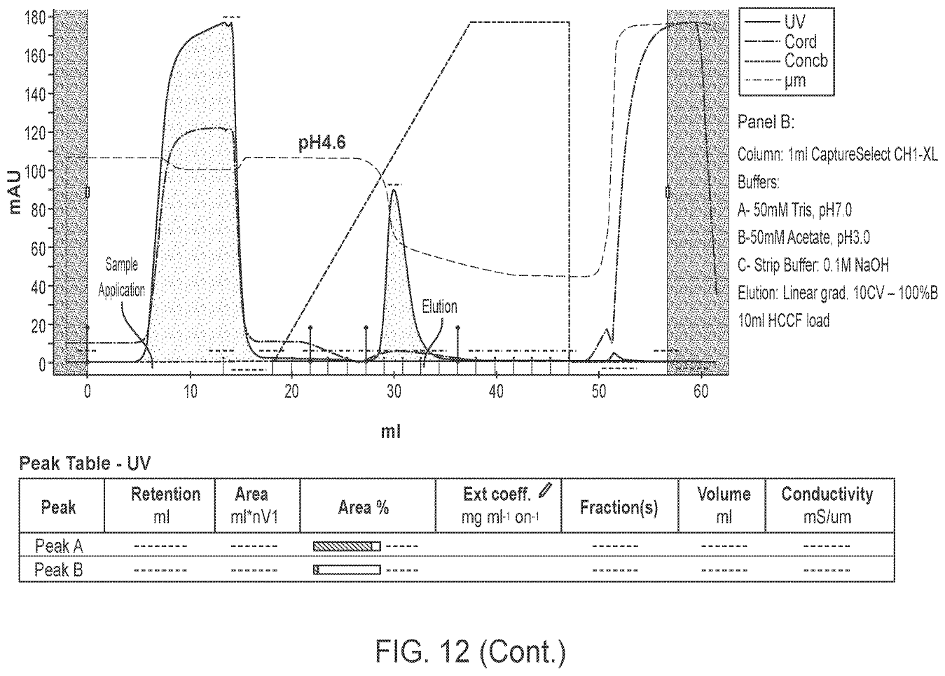

A comparison of the elution pH of the capture media is provided in FIG. 12. FIG. 12 demonstrates that using CH1-XL resin as a first capture step (instead of Protein A) allows a higher pH elution at pH 4.6, as compared to Protein A capture and elution at pH 3.3. These gentler elution conditions contribute to reduced antibody aggregation in the elution pool. The parameters as shown in FIG. 12 Panel A were as follows: Column: 1 ml MabSelect™ SuRe™, GE Healthcare Life Sciences; Load: 10 mL HCCF; Equilibration/Wash Buffer: 50 mM Tris, pH 7.0, 50 mM Acetate, pH 3.0; Strip Buffer: 0.1M NaOH; Elution: linear grad. 10 CV-100% B. The parameters as shown in FIG. 12 Panel B: Column: 1 mL CaptureSelect CH1-XL™; Load: 10 mL HCCF; Equilibration/Wash Buffer: 50 mM Tris, pH 7.0, 50 mM Acetate, pH 3.0; Strip Buffer: 0.1M NaOH; Elution: linear grad. 10 CV-100% B.

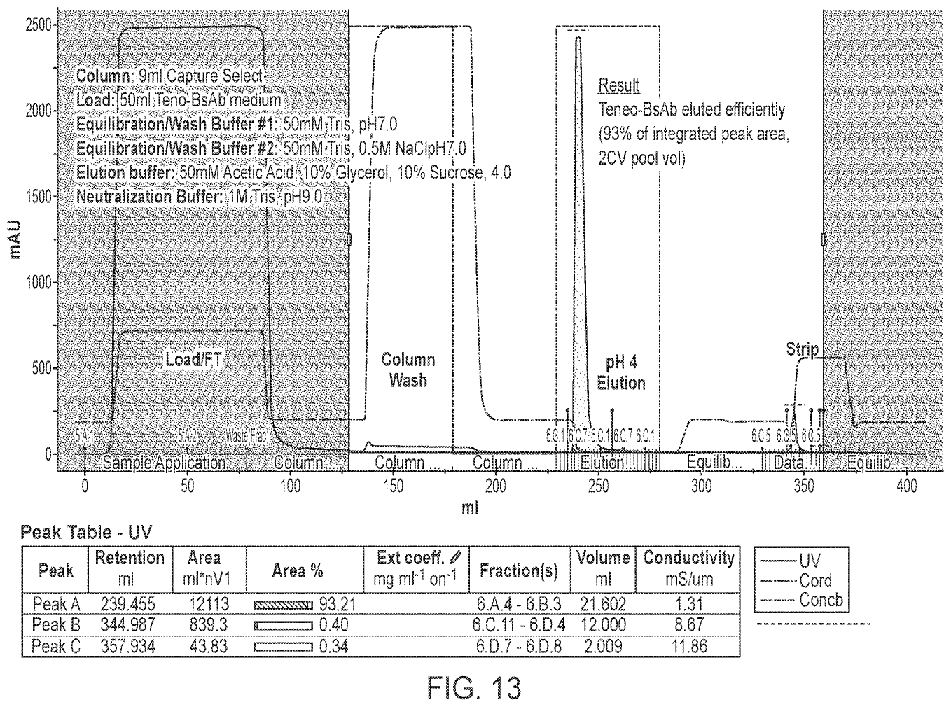

Elution of the BsAb from the CH1-XL resin was optimal using an elution buffer comprising 50 mM Acetic Acid, 10% glycerol and 10% sucrose, and having a pH of 4.0. Under these conditions, the BsAb eluted efficiently, with 93% of the integrated peak area present in a 2 CV pool volume. FIG. 13. CaptureSelect™ parameters were as follows: Column: 9 mL CaptureSelect; Load: 50 mL BsAb medium; Equilibration/Wash Buffer #1: 50 mM Tris, pH 7.0; Equilibration/Wash Buffer #2: 50 mM Tris, 0.5 M NaCl pH 7.0; Elution Buffer: 50 mM Acetic Acid, 10% glycerol, 10% sucrose, pH 4.0; Neutralization Buffer: 1M Tris, pH 9.0.

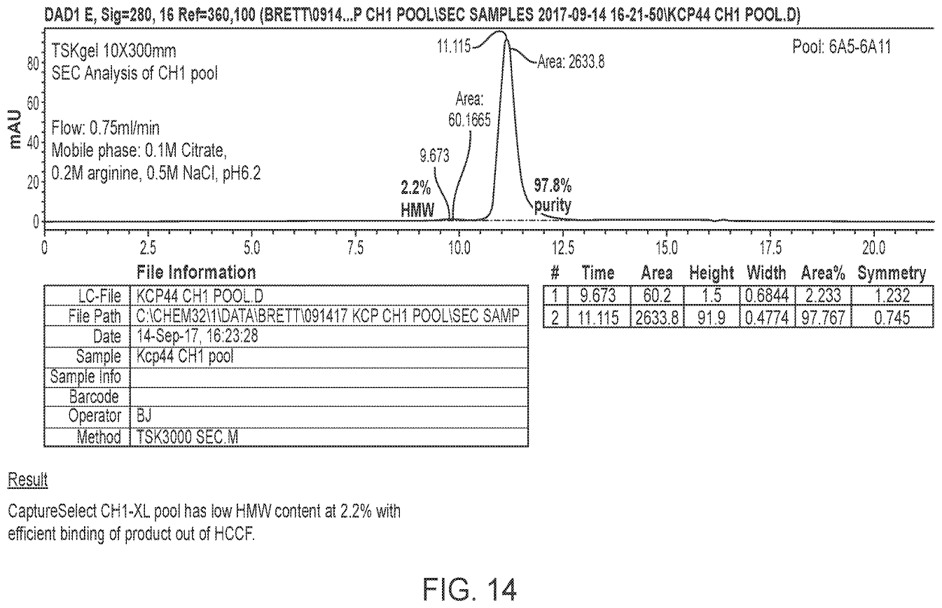

Further analysis of the CH1-XL pool revealed minimal BsAb aggregates (HMW content at 2.2% with efficient binding of BsAb product out of HCCF). FIG. 14. The parameters as shown in FIG. 14 were as follows: TSKgel 10×300 mm; Flow: 0.75 ml/min; Mobile Phase: 01M Citrate; 0.2M arginine, 0.5 M NaCl, pH 6.2. As such, in one preferred embodiment, a method for purifying a BsAb comprises a CH1-XL chromatography step, wherein the CH1-XL elution buffer comprises 50 mM Acetic Acid, 10% glycerol and 10% sucrose, and has a pH of 4.0.

Dynamic binding capacity of the CH1-XL resin was investigated, and the results are provided in FIG. 15. As shown in FIG. 15, the parameters were 1 mL CH1-XL column (0.7×2.5 cm); Load: purified BsAb 5 mg/ml; Residence times: 1, 2, 4, 8 minutes; 10% breakout before elution; P.C.: by 280 nm of pool. The results demonstrate that the dynamic binding capacity reached a plateau at 4 minutes, with a value of 9.3 mg/mL. Subsequent pilot scale work using HCCF demonstrated the potential to increase load density up to 19 mg/mL, such as about 10, 11, 12, 13, 14, 15, 16, 17, or about 18 mg/mL. As such, in some embodiments of the subject methods, a CH1-XL chromatography step comprises a load density that ranges from about 9 to about 19 mg/mL, such as about 10, 11, 12, 13, 14, 15, 16, 17, or about 18 mg/mL.

A schematic flow diagram of a manufacturing process that can be used to produce a BsAb in accordance with embodiments of the invention is provided in FIG. 16. The flow diagram shows representative upstream and downstream unit operations. An analysis of the BsAb species found at each stage of the purification process is provided in FIG. 17. Lane 1: molecular weight standards; Lane 2: HCCF 5 μl; Lane 3: CH1 Flow through 5 μl; Lane 4: CH1-XL1 pool 2 μg; Lane 5: Purification step 2—pool 2 μg; Lane 6: Purification step 3—pool 2 μg; Lane 7: molecular weight standards; Lane 8: CH1 pool 2 μg reduced; Lane 9: Purification step 2 reduced—pool 2 μg; Lane 10: Purification step 3 reduced—pool 2 μg. The parameters were as follows: NuPage 4-12% Bis-Tris gel; MES running buffer; InstantBlue stain (Expedeon); Page Ruler Prestained Protein Ladder; Protein load: 2 μg/lane; Run conditions: 35 min., 200V, 120 mA, 25 watts. The results demonstrate removal of the TAA homodimer species after the CH1-XL chromatography step. Overall yield for a manufacturing processes in accordance with embodiments of the invention ranged from about 70% to about 90%, such as about 75%, 80%, or about 85%.

### Example 2: Purification of a Bispecific Antibody Comprising Heavy Chain-Only Binding Units

A bispecific antibody comprising first and second binding units each comprising a heavy chain variable region of a heavy chain-only antibody is purified from a mixture comprising the antibody according to the methods described herein. The mixture comprising the bispecific antibody is contacted with a first affinity chromatography material, thereby immobilizing the antibody. The antibody is eluted with an elution buffer comprising an anti-aggregation composition comprising polyols as described herein, thereby reducing aggregation of the bispecific antibody in the elution pool.

### Example 3: Purification of a Bispecific Antibody Comprising Heavy Chain/Light Chain Binding Units

A bispecific antibody comprising first and second binding units each comprising a heavy chain variable region of an antibody and a light chain variable region of an antibody is contacted with a first affinity chromatography column, thereby immobilizing the antibody. The antibody is eluted with an elution buffer comprising an anti-aggregation composition comprising polyols as described herein, thereby reducing aggregation of the bispecific antibody in the elution pool.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for purifying a multispecific IgG antibody from a mixture by affinity chromatography, the method comprising:
immobilizing the multispecific IgG antibody from said mixture on a first affinity chromatography column having binding specificity to a heavy chain constant domain of said IgG antibody; and
eluting the multispecific antibody from the first affinity chromatography column with an elution buffer comprising an anti-aggregation composition to purify the multispecific antibody from the mixture, wherein the anti-aggregation composition comprises one or more polyols.

2. The method of claim 1, wherein the one or more polyols are selected from the group consisting of: mannitol, glycerol, sucrose, trehalose, and combinations thereof.

3. The method of claim 2, wherein the one or more polyols have a concentration that ranges from about 5% to about 25% w/v.

4. The method of claim 3, wherein the one or more polyols comprise glycerol, having a concentration that ranges from about 5% to about 15% w/v.

5. The method of claim 4, wherein the glycerol has a concentration of about 10% w/v.

6. The method of claim 3, wherein the one or more polyols comprise sucrose, having a concentration that ranges from about 5% to about 15% w/v.

7. The method of claim 6, wherein the sucrose has a concentration of about 10% w/v.

8. The method of claim 3, wherein the elution buffer comprises about 10% glycerol and about 10% sucrose w/v.

9. The method of claim 1, wherein the affinity chromatography column comprises a protein A chromatography resin.

10. (canceled)

11. The method of claim 10, wherein the elution buffer comprises citrate in a concentration that ranges from about 20 mM to about 30 mM.

12. (canceled)

13. The method of claim 9, wherein the elution buffer has a pH that ranges from about 3.2 to about 4.2.

14. The method of claim 13, wherein the elution buffer has a pH that ranges from about 3.4 to about 3.8.

15. (canceled)

16. The method of claim 9, wherein the elution buffer comprises about 25 mM citrate, about 10% glycerol, and about 10% sucrose, and wherein the elution buffer has a pH of about 3.6.

17. The method of claim 1, wherein the affinity chromatography column comprises a domain-specific chromatography resin that binds to a CH1 domain of the IgG antibody.

18. (canceled)

19. The method of claim 18, wherein the elution buffer comprises acetic acid in a concentration that ranges from about 45 mM to about 55 mM.

20. (canceled)

21. The method of claim 17, wherein the elution buffer has a pH that ranges from about 3.4 to about 4.4.

22. The method of claim 21, wherein the elution buffer has a pH that ranges from about 3.8 to about 4.2.

23. (canceled)

24. The method of claim 17, wherein the elution buffer comprises about 50 mM acetic acid, about 10% glycerol, and about 10% sucrose, and wherein the elution buffer has a pH of about 4.0.

25. A method of reducing aggregation of a multispecific IgG antibody in an elution pool from an affinity chromatography procedure, the method comprising:
immobilizing the multispecific IgG antibody on a protein A affinity chromatography column; and
eluting the multispecific IgG antibody from the protein A affinity chromatography column with an elution buffer comprising 25 mM citrate, 10% glycerol, and 10% sucrose w/v, wherein the elution buffer has a pH of 3.6.

26. A method of reducing aggregation of a multispecific IgG antibody in an elution pool from an affinity chromatography procedure, the method comprising:
immobilizing the multispecific IgG antibody on an affinity chromatography column comprising a domain-specific chromatography resin that has binding affinity to a CH1 domain of the multispecific IgG antibody; and
eluting the multispecific IgG antibody from the affinity chromatography column with an elution buffer comprising 50 mM acetic acid, 10% glycerol and 10% sucrose, wherein the elution buffer has a pH of 4.0.

27.-35. (canceled)
